(19) United States
(12) Patent Application Publication
(10) Pub. No.: US 2009/0181048 A1
(43) Pub. Date: Jul. 16, 2009

(54) CANCER DRUG DELIVERY USING MODIFIED TRANSFERRIN

(75) Inventors: Daniel T. Kamei, Monterey Park, CA (US); Bert J. Lao, Los Angeles, CA (US); Wen-Lin P. Tsai, Carlsbad, CA (US); Foad Mashayekhi, Los Angeles, CA (US); Edward A. Pham, Midway City, CA (US); Anne B. Mason, Charlotte, VT (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/134,922

(22) Filed: Jun. 6, 2008

Related U.S. Application Data
(60) Provisional application No. 60/942,794, filed on Jun. 8, 2007.

(51) Int. Cl. A61K 39/05; C07K 14/79; C12N 9/96

(52) U.S. Cl. 424/197.11; 530/394; 435/188

(57) ABSTRACT

The present invention provides transferrin (Tf) conjugates of anti-cancer agents with increased cellular association and increased cellular internalization. The present invention also provides methods of treating cancer comprising administration of a Tf conjugate with increased cellular association to a subject with cancer. The present invention additionally provides methods of making, as well as screening for, Tf conjugates with increased cellular association or cellular internalization. The present invention also provides Tf conjugates with increased cellular association and internalization for delivering nucleic acids to cancer cells.

### CROSS-REFERENCES TO RELATED APPLICATIONS

The present application claims priority to U.S. Ser. No. 60/942,794, filed Jun. 8, 2007, herein incorporated by reference in its entirety.

### STATEMENT AS TO RIGHTS TO INVENTIONS MADE UNDER FEDERALLY SPONSORED RESEARCH OR DEVELOPMENT

This invention was made with Governmental support of Grant No. DK021739 awarded by the National Institutes of Health. The Government has certain rights in this invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

One of the major challenges of treating cancer is avoiding drug toxicity associated with non-cancer cell drug interactions. These off target associations cause complications ranging from inflammation to the death of the patient. One way of avoiding these problems is to target cancer therapeutics specifically to cancer cells. A common approach to achieve this is to conjugate anti-cancer agents to antibodies or functional fragments thereof that target cancer-specific antigens overexpressed on the surface of neoplastic cells. Although these therapeutics hold some promise, antibody therapy can still result in significant levels of non-specific cellular association.

In order to further overcome this obstacle, the serum iron transport protein transferrin (Tf) has been investigated as a potential drug carrier. Conjugation of anticancer agents to Tf allows for specific targeting to cancer cells, since the transferrin receptor (TfR) is overexpressed in a broad range of cancers (Cazzola et al., Blood. 1990; 75(10):1903-19; Reizenstein, Med Oncol Tumor Pharmacother. 1991; 8(4):229-33). Specific targeting of drugs to cancer cells with Tf may help alleviate nonspecific toxicity associated with chemotherapy and radiation treatments (Saul et al., J Control Release. 2006; 114(3):277-87; Kreitman, Aaps J. 2006; 8(3):E532-51). Tf conjugates of cytotoxins including methotrexate (MTX), artemisinin, and diphtheria toxin (DT) have been reported, as well as Tf conjugates with novel payloads such as liposomally encapsulated drugs and siRNA (Lim and Shen, Pharm Res. 2004; 21(11): 1985-92; Lai et al., Life Sci. 2005; 76(11): 1267-79; Johnson et al., J Biol Chem. 1988; 263(3): 1295-300; Hu-Lieskovan et al., Cancer Res. 2005; 65(19): 8984-92; Tros et al., J Drug Target. 2006; 14(8):527-35; Maruyama et al., J Control Release. 2004; 98(2):195-207; Chin et al., J Control Release. 2006; 112(2):199-207).

The use of Tf conjugates for cancer therapy is currently being assessed in clinical trials. For example, Tf conjugates of CRM107, a point mutant of DT with reduced nonspecific binding, are being studied as treatment for malignant gliomas. Results of a Phase II trial indicated complete and partial tumor response in 35% of patients treated with the Tf-CRM107 conjugate (Weaver and Laske, J Neurooncol. 2003; 65(1):3-13).

Although Tf has been extensively investigated as a potential delivery agent for cancer therapeutics, the rapid recycling of Tf through the endocytic TfR pathway may significantly limit its efficiency as a drug carrier (Lim and Shen, Pharm Res. 2004; 21(11):1985-92). Tf is recycled back into the bloodstream as apo-Tf, which has a non-detectable binding affinity for TfR (Lebron et al., Cell. 1998; 93(1): 111-23). Rebinding of iron by Tf can be a variable and inefficient process, and therefore in models of Tf trafficking, recycled Tf is often assumed to be lost (Yazdi and Murphy, Cancer Res. 1994; 54(24):6387-94; Ciechanover et al., J Biol Chem. 1983; 258(16):9681-9). Thus, the window of drug delivery for a Tf conjugate may well be limited to one passage through a cell.

Furthermore, studies suggest that the translocation of drug from the conjugate into the cytosol is frequently the rate-limiting step of the overall drug delivery process (Yazdi et al., Cancer Res. 1995; 55(17):3763-71). For example, it has been estimated that in the case of Tf conjugates of the gelonin cytotoxin that for every ten million conjugates that are recycled, only one molecule of gelonin is actually delivered into the cell. Therefore, it appears unlikely that any given gelonin conjugate trafficking once through the cell will deliver its drug and achieve its intended purpose.

To address this inefficiency, alternate TfR ligands with different trafficking properties than those of Tf have been investigated, such as TfR mAbs and Tf oligomers (Yazdi et al., Cancer Res. 1995; 55(17):3763-71; Lim and Shen, Pharm Res. 2004; 21(11):1985-92). Because these ligands continue to utilize the TfR pathway, they maintain specificity towards cancer cells which overexpress TfR. However, unlike Tf, these ligands appear to favor intracellular degradation; thus, they tend to be routed to cellular lysosomes instead of being recycled. This has the effect of increasing the length of time the ligand remains associated with the cell, thereby increasing the probability of delivering the drug. Indeed, MTX conjugates of Tf oligomers were shown to be more cytotoxic than MTX conjugates of native Tf (Lim and Shen, Pharm Res. 2004; 21(11):1985-92).

Although routing conjugate traffic to the lysosome appears effective for MTX, which requires release from Tf to be active, lysosomal degradation may adversely affect the effectiveness of protein drugs such as DT and CRM107. In fact, TfR mAb conjugates of CRM107 are less cytotoxic than Tf conjugates of CRM107, though the reasons for this are not clear (Wenning et al., Biotechnol Bioeng. 1998; 57(4):484-96).

The current invention satisfies a need in the art for Tf conjugates with increased levels of cellular association and cellular internalization.

### BRIEF SUMMARY OF THE INVENTION

The present invention provides transferrin (Tf) conjugates of anti-cancer agents with increased cellular association and increased cellular internalization. The present invention also provides methods of treating cancer comprising administration of a Tf conjugate with increased cellular association to a subject with cancer. The present invention additionally provides methods of making, as well as screening for, Tf conjugates with increased cellular association or cellular internalization. The present invention also provides Tf conjugates with increased cellular association and internalization for delivering nucleic acids to cancer cells.

In one embodiment, the present invention provides Tf conjugates with decreased iron release kinetics. In a particular embodiment, the decreased iron release kinetics are a result of the substitution of the iron coordinating anion, carbonate, with a second anion. In one embodiment, the invention provides a Tf conjugate, wherein Tf binds oxalate rather than carbonate. In another embodiment, the decreased iron release kinetics are a result of one or more mutations in the amino acid sequence of Tf. In a particular embodiment, the Tf molecule comprises more than one mutation.

In one embodiment, the present invention provides Tf conjugates of mutant Tf, wherein the mutant Tf has decreased iron release kinetics compared to wild type Tf. In one embodiment, the Tf molecule comprises 2, 3, 4, 5, 6, 7, 8, 9, 10, 11, 12, 13, 14, 15, 16, 17, 18, 19, 20, 30, 40, 50, or more mutations. In another embodiment, the Tf molecule has at least 85% identity to SEQ ID NO:1. In yet another embodiment, the Tf molecule has at least 50%, 55%, 60%, 65%, 70%, 75%, 80%, 85%, 90%, 91%, 92%, 93%, 94%, 95%, 96%, 97%, 98%, 99%, or more amino acid sequence identity to SEQ ID NO:1. In a particular embodiment, the mutant Tf molecule retains about wild type binding affinity for the transferrin receptor (TfR). In another particular embodiment, the Tf molecule retains at least wild type binding affinity for the TfR. In yet another embodiment, the Tf molecule has at least 85%, 90%, 95%, or more amino acid sequence identity to the amino acid sequence of a Tf molecule with an accession number NP_{—}001054, NP_{—}598738, NP_{—}001013128, or XP_{—}001364584.

In one embodiment, the Tf mutation comprises a mutation at a residue selected from the group consisting of K206, R632, K534, and combinations thereof. In a particular embodiment, the Tf molecule comprises a mutation selected from the group consisting of K206E, R632A, K534A, and combinations thereof. In another embodiment, the Tf molecule comprises a mutation at residue K206. In a particular embodiment, the mutation at residue K206 is selected from Ala, Gly, Leu, Ile, Val, Pro, Asp, and Glu. In another particular embodiment, the mutation at residue R632 is selected from Ala, Gly, Leu, Ile, Val, and Pro. In yet another embodiment, the mutation at residue K534 is selected from Ala, Gly, Leu, Ile, Val, Pro. In another embodiment, the Tf mutation comprises a mutation at a residue selected from the group consisting of K206, K296, H349, K534, R632, D634, and combinations thereof.

In one embodiment, the present invention provides methods of treating cancer in a mammal by administering a Tf conjugate of an anticancer agent to said mammal, wherein the Tf conjugate has decreased iron release kinetics. In one embodiment, the mammal is a human, mouse, rat, hamster, guinea pig, rabbit or monkey. In a particular embodiment, the cancer being treated is brain cancer. In another particular embodiment, the brain cancer is a glioblastoma multiforme tumor.

In the current invention, we have applied a mathematical model of Tf/TfR cellular trafficking in order to identify methods of altering the trafficking pathway of Tf that increase its cellular association. We have identified the release rate of iron from Tf as a previously unidentified factor influencing the degree of cellular association. We demonstrate that Tf ligands modified to inhibit iron release associate with HeLa cells to a greater extent when compared to native Tf. Finally, we show that DT conjugates of iron release modified Tf ligands are significantly more cytotoxic than DT conjugates of native Tf in an in vitro cell-killing assay.

### BRIEF DESCRIPTION OF THE DRAWINGS

FIG. 1 Shows a schematic of the Tf/TfR trafficking pathway.

FIG. 2 Shows an illustration of Tf properties varied in the model.

FIG. 3 Shows predicted responses in cellular association to changes in Tf parameters. Dashed lines indicate native Tf values for each property. Note change in scale for plots 3C and 3D.

FIG. 4 Shows internalized Tf/cell for oxalate Tf vs. native Tf at concentrations of 0.1, 1, and 10 nM. Error bars represent the standard deviation from an average of three measurements.

FIG. 5 Shows a cytotoxicity comparison between DT conjugates of oxalate Tf vs. native Tf. Error bars represent standard error from an average of four experiments.

FIG. 6 Shows proposed alternative trafficking pathway for oxalate Tf.

FIG. 7 Shows a sensitivity analysis for the k_{FeTf,TfR} and k_{FeTf,TfR,r} equilibrium parameters.

FIG. 8 Shows a sensitivity analysis for the k_{endo}, k_{endo,r}, and kᵢₙₜ equilibrium parameters.

FIG. 9 Shows the 1.2 Å resolution crystal structure of the N-terminal lobe of human serum Tf coordinating Fe and oxalate, PDB ID: 1RYO.

FIG. 10 Shows the superposition of the high-resolution crystal structures of the N-terminal lobe of human serum Tf coordinating Fe with both an oxalate anion (PDB ID: 1RYO) and a carbonate anion (PDB ID: 1A8E).

FIG. 11 Shows a close-up of the Fe binding pocket for the superposition of the N-lobes of human Tf PDB ID: 1RYO, with oxalate, and 1A8E, with carbonate.

FIG. 12 Shows the 2.7 Å resolution crystal structure of the full-length human Tf protein in the apo-form.

FIG. 13 Shows the modeled structure of the Tf, cartoon representation, -TfR, space-filling model, complex, PDB ID: 1SUV.

FIG. 14 Internalized Tf/cell for oxalate N-His hTf NG vs. N-His hTf NG at a 1 nM concentration. Error bars represent the standard deviation from an average of three measurements.

FIG. 15 Internalized Tf/cell for the three recombinant Tf mutants vs. N-his hTf NG at a 1 nM concentration. Error bars represent the standard deviation from an average of three measurements.

SEQ ID NO:1 is the amino acid sequence of human serum Tf, NP_{—}001054.

### DETAILED DESCRIPTION OF THE INVENTION

Transferrin (Tf) conjugates of CRM107 are currently being tested in clinical trials for treatment of malignant gliomas. However, the rapid cellular recycling of Tf limits its efficiency as a drug carrier. We have developed a mathematical model of the Tf/TfR trafficking cycle and have identified the Tf iron release rate as a previously unreported factor governing the degree of Tf cellular association. The present invention provides Tf proteins and conjugates with reduced cellular recycling rates. In one embodiment, the current invention provides Tf conjugates with reduced iron release kinetic properties.

The release of iron from Tf is inhibited by replacing the synergistic carbonate anion with oxalate. Trafficking patterns for oxalate Tf and native Tf are compared by measuring their cellular association with HeLa cells. The amount of Tf associated with the cells is an average of 51% greater for oxalate Tf than for native Tf over a two hour period at Tf concentrations of 0.1 nM and 1 nM. Importantly, diphtheria toxin (DT) conjugates of oxalate Tf are more cytotoxic against HeLa cells than conjugates of native Tf. Conjugate IC₅₀ values were determined to be 0.06 nM for the oxalate Tf conjugate vs. 0.22 nM for the native Tf conjugate. Thus, the inhibition of Tf iron release improves the efficacy of Tf as a drug carrier through increased association with cells expressing TfR. One of skill in the art will recognize that other well known divalent anions can also be used in accordance with the present invention. Specifically, one of skill in the art will instantly recognize other suitable anions that will decrease the iron release kinetics of Tf.

In addition to Tf, other ligands which bind TfR have been used to target therapeutics to cancer cells, including TfR monoclonal antibodies (mAbs) and Tf oligomers (Lim and Shen, Pharm Res. 2004; 21(11): 1985-92; Yazdi et al., Cancer Res. 1995; 55(17):3763-71). Work from the Murphy laboratory has explored the principles governing the effectiveness of different cytotoxin conjugates targeting TfR (Yazdi et al., Cancer Res. 1995; 55(17):3763-71; Yazdi and Murphy, Cancer Res. 1994; 54(24):6387-94; Wenning et al., Biotechnol Bioeng. 1998; 57(4):484-96). Using in vitro cell experiments and mathematical modeling, Murphy showed that the cellular trafficking of the ligand is an important factor. Specifically, it was demonstrated that the duration of cellular trafficking is correlated with the effectiveness of drug delivery. For example, the trafficking of TfR mAbs is somewhat different than Tf, because the mAbs are more prone to intracellular degradation than to being recycled to the cell surface. This increases the cellular association of TfR mAbs relative to Tf, and as a result, gelonin conjugates of TfR mAbs show increased cytotoxicity over gelonin conjugates of Tf (Yazdi et al., Cancer Res. 1995; 55(17):3763-71). Similarly, oligomerization of Tf increases intracellular degradation in comparison to monomeric Tf. Consequently, MTX conjugates of Tf oligomers show greater cytotoxicity than MTX conjugates of monomeric Tf (Lim and Shen, Pharm Res. 2004; 21(11): 1985-92).

Therefore, the effectiveness of cytotoxin conjugates may be enhanced by identifying ligands for TfR which exhibit an increased degree of cellular association. One strategy is to engineer Tf in order to modify its normal trafficking behavior such that its cellular association is increased. The general principle of modifying cellular trafficking behavior to achieve a beneficial effect has been successfully applied to other systems. For example, a mutated version of granulocyte colony stimulating factor (GCSF) which favors the cellular recycling pathway results in a significantly extended GCSF half-life (Sarkar et al., Nat Biotechnol. 2002; 20(9):908-13). In addition, IgG antibodies with mutated Fc regions resulting in greater binding affinities for the FcRn receptor in the endosome show an increase in cellular recycling, leading to longer half-lives in vivo (Hinton et al., J Biol Chem. 2004; 279(8):6213-6).

To test the prediction that lowering the iron release rate of Tf would result in increased cellular association and internalization, Tf ligands with reduced iron release rates were constructed by replacing the synergistic carbonate anion with oxalate. Oxalate lowers the iron release rate of Tf by increasing the structural stability of the Tf iron binding sites (Ciechanover et al., J Biol Chem. 1983; 258(16):9681-9) and because it has a lower pKₐ than carbonate (Johnson et al., J Biol Chem. 1988; 263(3):1295-300). Oxalate is present normally in the human circulation at micromolar concentrations, and hence its presence would not be expected to be problematic for in vivo administration (Costello and Landwehr, Clin Chem. 1988; 34(8):1540-4). Additionally, the oxalate complex of Tf would not be predicted to elicit an immune response. Tf ligands modified with oxalate were found to associate with HeLa cells an average of 51% more than native Tf at concentrations of 0.1 and 1 nM.

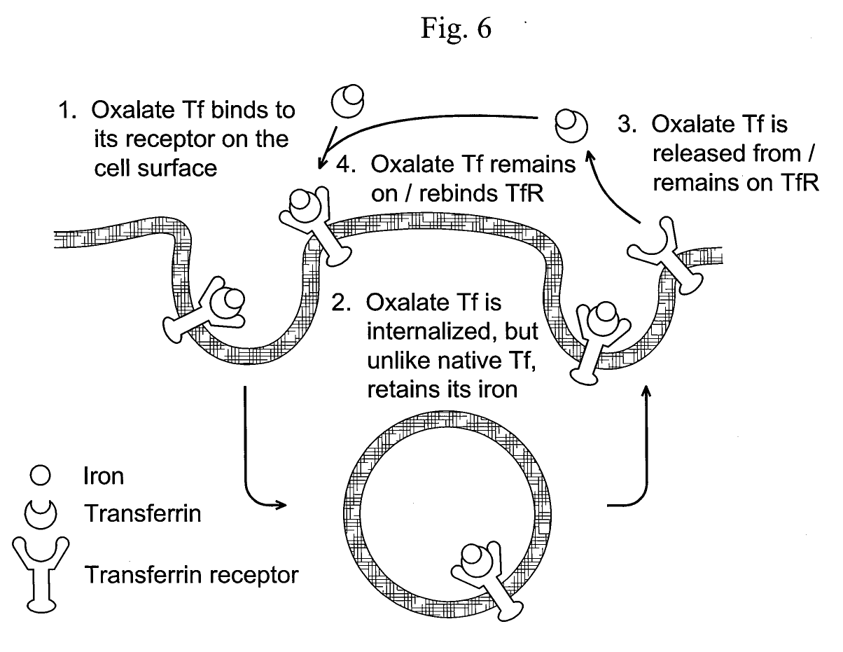

These results suggest that if the iron release rate of Tf is lowered such that Tf holds its iron upon recycling to the cell surface, then Tf may retain its ability to either bind to or remain associated with TfR, and may thus be reinternalized. This appears to enable Tf to undergo multiple cycles through the cell without having to rebind iron, thereby altering the normal trafficking pathway of Tf to increase its cellular association. This proposed alternative trafficking pathway is illustrated in FIG. 6.

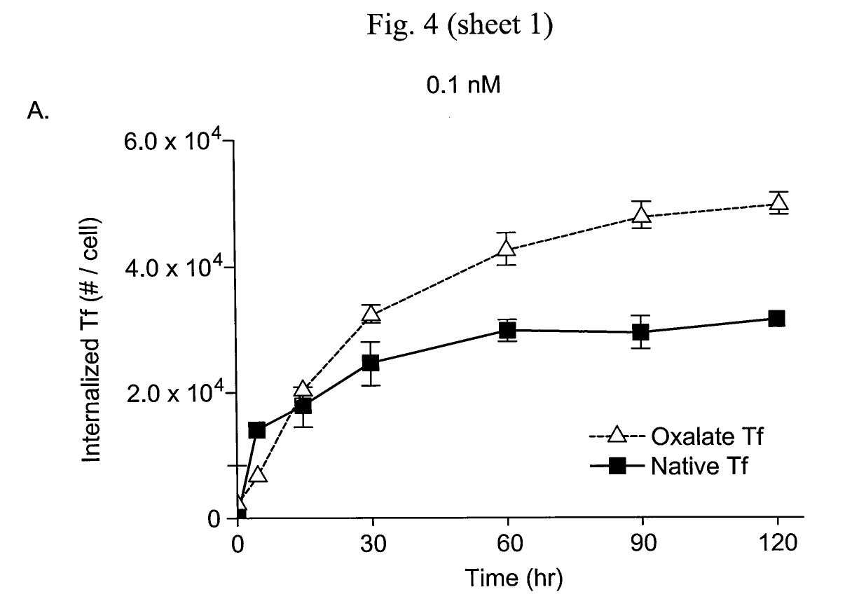

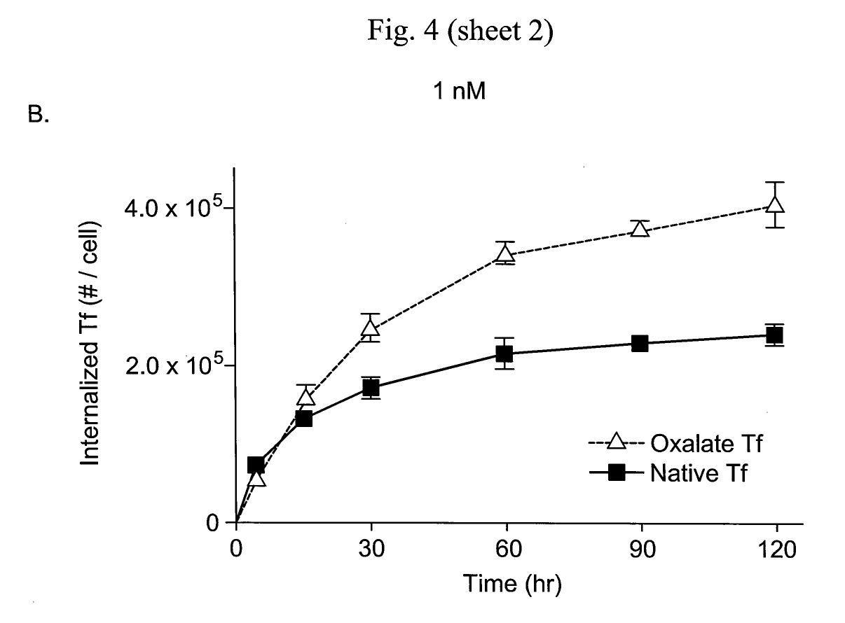

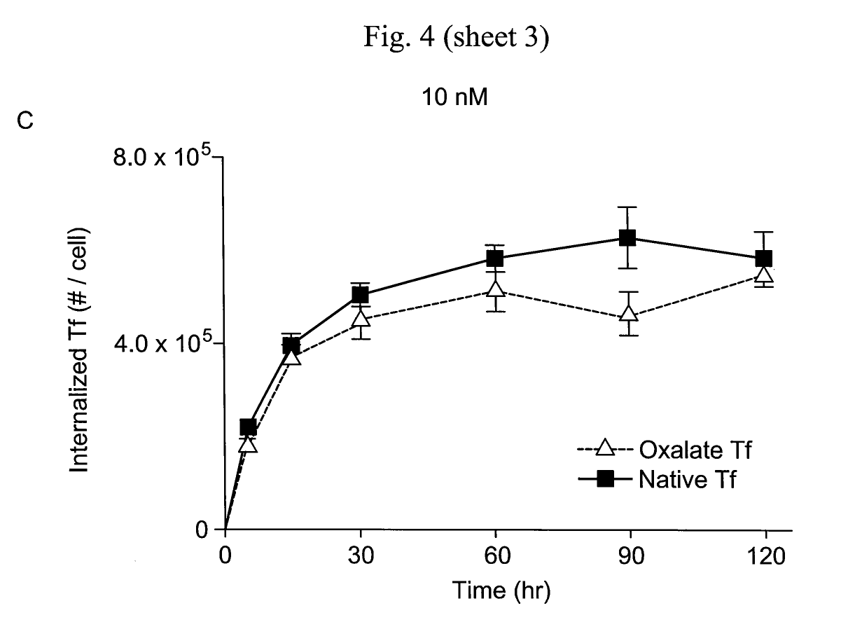

We produced conjugates of Tf with the DT cytotoxin to test whether the observed increase in cellular association would translate into improved drug carrier efficacy. HeLa cells were selected for the cytotoxicity assay due to their high expression of transferrin receptors (5.4×10⁵ receptors/cell) (Yazdi and Murphy, Cancer Res. 1994; 54(24):6387-94). In future work, additional cell lines expressing TfR, such as the K562 and HL60 human leukemia cell lines, may be tested to broaden the applicability of the conjugates (Berczi et al., Arch Biochem Biophys. 1993; 300(1):356-63). DT was selected as a cytotoxin since the effective concentration range of DT (IC₅₀˜0.1 nM) (Johnson et al., J Biol Chem. 1988; 263(3): 1295-300) is consistent with the concentration range in which increases in cellular association were observed in the cellular trafficking assay (0.1 and 1 nM, FIG. 4). This is in contrast to cytotoxins such as adriamycin, whose effective concentration range is considerably higher (IC₅₀˜1 μM) (Berczi et al., Arch Biochem Biophys. 1993; 300(1):356-63). Thus, oxalate Tf conjugates with these cytotoxins would not be expected to display increased efficacy, since differences in cellular association between oxalate Tf and native Tf diminish as concentrations rise to 10 nM (FIG. 4C). In the results of our cytotoxicity assay, oxalate Tf conjugates of DT showed greater cytotoxicity than native Tf conjugates of DT against HeLa cells, with an IC₅₀ value of 0.06 nM compared to 0.22 nM for native Tf conjugates.

Tumor extracellular pH was measured by Gerweck et al. in mice and found to be 6.77, which is somewhat more acidic than the pH of the bloodstream, 7.4 (Gerweck et al., Mol Cancer Ther. 2006; 5(5): 1275-9). Intracellular tumor pH was found to be similar to normal tissue. The acidic extracellular pH of tumors was not accounted for in our model, which was designed to guide our in vitro experiments. We would expect the acidic tumor pH to be problematic if it significantly promoted conversion of holo-Tf to apo-Tf before binding of Tf to TfR on the tumor surface. Although apo-Tf has a high affinity for TfR at the acidic pH of the endosome, this high affinity is due in part to protonation of histidines at its TfR binding site (Giannetti et al., Structure (Camb). 2005; 13(11):1613-23). Since histidines have a pKₐ of 6.4, we would not expect these histidines to be protonated at an extracellular pH of 6.77. Therefore, it is important to consider whether a pH of 6.77 would significantly promote iron release from Tf. The retention of iron as a function of pH has been studied for both native Tf and oxalate Tf (Halbrooks et al., J Mol Biol. 2004; 339(1):217-26). Following a one week equilibration period, it was found that oxalate Tf retained over 90% of its iron at a pH of 6.77, while native Tf retained over 80%. Thus, we would expect the majority of Tf to remain as holo-Tf in the acidic extracellular environment of the tumor and retain its ability to bind to TfR on the tumor surface.

The high concentration of endogenous holo-Tf in the bloodstream (3-6 μM) (Johnson and Enns, Blood. 2004; 104(13):4287-93) likely precludes the intravenous administration of Tf conjugates in vivo, as any administered conjugates would have difficulty competing with endogenous Tf for binding to TfR. Indeed, intravenously administered imaging agents conjugated with Tf were unable to specifically target tumors in mice (Aloj et al., J Nucl Med. 1999; 40(9): 1547-55). Rather, high concentrations of endogenous Tf will likely necessitate the administration of Tf conjugates within the vicinity of a tumor, as in clinical trials of a Tf-CRM107 conjugate for malignant gliomas (Weaver and Laske, J Neurooncol. 2003; 65(1):3-13).

Inhibiting Tf iron release through replacement of carbonate with oxalate may provide a relatively simple and inexpensive method to improve the efficacy of Tf drug carriers through increased association with cancer cells. However, increasing the cellular association of a TfR ligand might also result in an undesired increase in cytotoxicity to normal cells, since TfR is ubiquitously expressed (Hentze and Muckenthaler, Cell. 2004; 117(3):285-97). This may be expected to be the case for ligands such as the TfR mAbs 5E9 and OKT9, which bind to a site on TfR distinct from that of Tf and thus do not compete with endogenous Tf (Wenning et al., Biotechnol Bioeng. 1998; 57(4):484-96; Aloj et al., J Nucl Med. 1999; 40(9):1547-55). However, since replacement of carbonate with oxalate does not significantly affect the binding affinity of Tf for TfR, conjugates of oxalate Tf which do not reach their intended target may be unable to bind normal cells due to high levels of endogenous Tf.

In one embodiment, the present invention provides mutant Tf conjugates with decreased iron release kinetics. Many Tf mutants with decreased iron release kinetics are known in the art. Examples of residues that can be mutated to achieve the desired result include, without limitation, K206, K296, H349, H350, K534, R632, D634, and combinations thereof. One of skill in the art will appreciate that other mutations can be designed or screened for in order to decrease the iron release kinetics of Tf.

A number of Tf ligands with inhibited iron release rates, that are particularly well suited for use in the present invention, have been generated through site-directed mutagenesis of their iron binding sites. The iron release rates of these Tf mutants are presented in Tables 1-3. The experimental conditions under which the data were obtained are indicated below in the table headings. These data were obtained in vitro in the presence of iron chelators, and are meant only as indicators of the relative, qualitative differences in iron release rates between the different Tf ligands.

TABLE 1
At pH 5.6, 4 mM of the EDTA chelator was used. At pH
7.4, 12 mM of the Tiron chelator was used (Halbrooks
et al., J Mol Biol 339(1): 217-26 (2004); Halbrooks
et al., Biochemistry 42(13): 3701-3707 (2003)).

| | N-lobe, pH | C-lobe, pH | N-lobe, pH | C-lobe, pH |
|---|---|---|---|---|
| | 5.6 (min⁻¹) × | 5.6 (min⁻¹) × | 7.4 (min⁻¹) × | 7.4 (min⁻¹) × |
| | 10⁻³ | 10⁻³ | 10⁻³ | 10⁻³ |
| Wild- | 2610.0 ± 33.4 | 126.9 ± 5.6 | 54.7 ± 3.3 | 40.4 ± 1.3 |
| Type Tf | | | | |
| K206E | 0.60 ± 0.02 | 139.6 ± 6.0 | no release | 37.0 ± 1.7 |
| K206E/ | 0.57 ± 0.04 | 5.2 ± 0.05 | no release | no release |
| K534A | | | | |
| K206E/ | 0.60 ± 0.05 | 1.5 ± 0.09 | no release | no release |
| R632A | | | | |

TABLE 2
At pH 5.6, 4 mM of the EDTA chelator was used. At
pH 7.4, 12 mM of the Tiron chelator was used (He
et al., Biochemistry 38(30): 9704-9711 (1999)).

| | Fe removal by EDTA, | Fe removal by Tiron, |
|---|---|---|
| | pH 5.6 (min⁻¹) | pH 7.4 (min⁻¹) |
| Wild-Type Tf | 4.09 ± 0.16 | 2.25 ± 0.09 × 10⁻² |
| K206E | 1.61 ± 0.09 × 10⁻⁴ | 6.42 ± 0.04 × 10⁻⁵ |
| K206Q | 1.05 ± 0.03 × 10⁻² | 8.82 ± 0.11 × 10⁻⁵ |
| K296E | 1.49 ± 0.08 × 10⁻² | 1.28 ± 0.08 × 10⁻⁴ |
| K296Q | 3.75 ± 0.21 × 10⁻² | 2.04 ± 0.13 × 10⁻⁴ |
| K206E/K296E | 8.98 ± 0.42 × 10⁻² | 6.11 ± 0.26 × 10⁻⁴ |

TABLE 3
500 μM of a pyrophosphate chelator was used (Steinlein
et al., Biochemistry 37(39): 13696-13703 (1998))

| | pH 6.5 | pH 5.7 | pH 5.2 | pH 5.0 |
|---|---|---|---|---|
| | (min⁻¹) | (min⁻¹) | (min⁻¹) | (min⁻¹) |
| Wild- | 2.64 ± 0.34 | 11.4 ± 3.8 | too fast | too fast |
| Type Tf | | | | |
| K206A/ | too slow | too slow | 0.087 ± 0.020 | 0.172 ± 0.020 |
| K296A | | | | |
| K206A | too slow | too slow | too slow | 0.018 ± 0.002 |
| K296A | too slow | too slow | too slow | 0.010 ± 0.002 |

In another embodiment of the present invention, further inhibition of Tf iron release can be achieved through alteration of the TfR binding interface. It has been previously reported that the binding of Tf to TfR stimulates the release of iron from Tf. Iron is released from unbound Tf on the order of days at acidic pH, but on the order of minutes when Tf is bound to TfR within the endosome. A study on the molecular mechanism of Tf iron release found that Tf histidine residues H349 and H350 were involved in the enhancement of Tf iron release upon binding to TfR (Giannetti et al., Structure 13(11): 1613-1623 (2005)). When each of these histidines were individually mutated to either alanine or lysine (H349A or H349K), the enhancement of iron release from binding to TfR was significantly reduced.

In one embodiment, the invention provides mutant Tf conjugates comprising mutations that decrease iron release kinetics by altering the TfR binding interface. In one embodiment, the Tf molecule comprises a mutation at either or both of H349 and H350. In a particular embodiment, the Tf molecule comprises a mutation at position 349 of SEQ ID NO:1, selected from the group of amino acids consisting of Ala, Gly, Ile, Leu, Val, and Pro. In another embodiment, the invention provides a Tf conjugate with a mutation at position 350 of SEQ ID NO:1 selected from the group consisting of Lys, Arg, Glu, Asp, Asn, and Gln.

The Tf conjugates of the present invention encompass native and recombinant Tf molecules. These Tf molecules may be glycosylated with wild type patterns of glycosylation, have reduced glycosylation, have improved patterns of glycosylation, or may not be glycosylated at all. The Tf peptides of the present invention may alternatively, or in addition, be further posttranslationally modified. Posttranslational modification may include natural modifications or non-natural modification. Examples of modifications suitable for use in the present invention include, without limitation, phosphorylation, glycosylation, methylation, alkylation, biotinylation, citrullination, deamidation, SUMOylation, NEDylation, PEGylation, palmitoylation, sugar moiety attachment, etc. In a particular embodiment, the Tf peptide is modified to increase its half-life in vivo.

In one embodiment, the present invention provides methods of treating a subject with cancer. In one embodiment, the method comprises administering to a subject with cancer a Tf conjugate of a cytotoxic drug or anti-cancer agent. In a particular embodiment, the Tf conjugate has decreased iron release kinetics as compared to native Tf conjugates. In another embodiment, the Tf conjugate comprises a diphtheria toxin. In a particular embodiment, the diphtheria toxin is CRM107. In an embodiment of the invention, the subject is a mammal, such as a human, mouse, rat, guinea pig, rabbit, monkey, or hamster. In another embodiment, the cancer being treated is brain cancer. Tf conjugates described elsewhere in the present application are particularly well suited for use in the methods of treatment described here.

Examples of anti-cancer agents or cytotoxic agents useful for the methods and conjugates of the present invention are well known in the art and include, without limitation, tamoxifen, toremifen, raloxifene, droloxifene, iodoxyfene, megestrol acetate, anastrozole, letrazole, borazole, exemestane, flutamide, nilutamide, bicalutamide, cyproterone acetate, goserelin acetate, luprolide, finasteride, herceptin, methotrexate, 5-fluorouracil, cytosine arabinoside, doxorubicin, daunomycin, epirubicin, idarubicin, mitomycin-C, dactinomycin, mithramycin, cisplatin, carboplatin, melphalan, chlorambucil, busulphan, cyclophosphamide, ifosfamide, nitrosoureas, thiotephan, vincristine, taxol, taxotere, etoposide, teniposide, amsacrine, irinotecan, topotecan, and epothilone; a tyrosine kinase inhibitor such as Iressa or OSI-774; an angiogenesis inhibitor; a diphtheria toxin, an EGF inhibitor; a VEGF inhibitor; a CDK inhibitor; a Her1/2 inhibitor and monoclonal antibodies directed against growth factor receptors such as erbitux (EGF) and herceptin (Her2). One of skill in the art will know of other acceptable agents that are useful for the conjugates and methods of the present invention.

Examples of anti-cancer agents acceptable for use in the present invention include, without limitation, alkylating agents, anti-metabolites, plant alkaloids and terpenoids, topoisomerase inhibitors, antineoplastics, hormone therapeutics, photosensitizers, kinase inhibitors, etc.

Examples of alkylating agents useful for conjugates and methods of the present invention include, without limitation, Cisplatin, Caroplatin, Oxaliplatin, Mechlorethamine, Cyclophophamide, Chlorambucil, Busulfan, Hexamethylmelamine, Thiotepa, Cyclophohphamine, Uramustine, Melphalan, Ifosfamide, Carmustine, Streptozocin, Dacarbazine, Temozolomide, etc.

Examples of anti-metabolite agents useful for conjugates and methods of the present invention include, without limitation, Aminopterin, Methotrexate, Pemetrexed, Raltitrexed, Cladribine, Clofarabine, Fludarabine, Mercaptopurine, Pentostatin, Thioguanine, Capecitabine, Cytarabine, Decitabine, Fluorouracil, Floxuridine, Gemcitabine, etc.

Examples of plant alkaloids and terpenoids useful for conjugates and methods of the present invention include, without limitation, Docetaxel, Larotaxel, Paclitaxel, Vinblastine, Vincristine, Vindesine, Vinorelbine, etc.

Examples of topoisomerase inhibitors useful for conjugates and methods of the present invention include, without limitation, Camptothecin, Topotecan, Irinotecan, Rubitecan, Etoposide, Teniposide, etc.

Examples of antineoplastics useful for conjugates and methods of the present invention include, without limitation, Daunorubicin, Doxorubicin, Epirubicin, Idarubicin, Mitoxantrone, Pixantrone, Valrubicin, Actinomycin, Bleomycin, Mitomycin, Plicamycin, etc.

Examples of photosensitizer agents useful for conjugates and methods of the present invention include, without limitation, Aminolevulinic acid, Methyl aminolevulinate, Porfimer sodium, Verteporfin, etc.

Examples of kinase inhibitors useful for conjugates and methods of the present invention include, without limitation, Axitinib, Bosutinib, Cediranib, Dasatinib, Erlotinib, Gefitinib, Imatinib, Lapatinib, Lestaurtinib, Nilotinib, Semaxanib, Sorafenib, Sunitinib, Vandetanib, Seliciclib, etc.

Examples of other anti-cancer agents useful for conjugates and methods of the present invention include, without limitation, Alitretinoin, Tretinoin, Aflibercept, Altretamine, Amsacrine, Anagrelide, Arsenic trioxide, Pegaspargase, Bexarotene, Bortezomib, Celecoxib, Denileukin diftitox, Elesclomol, Estramustine, Irofulven, Ixabepilone, Masoprocol, Mitotane, Oblimersen, Testolactone, Tipifarnib, Trabectedin.

The translation product of transferrin mRNA contains 698 amino acid residues, which is 19 amino acids longer than the molecule isolated from human plasma. The extra residues comprise a leader sequence or signal peptide which is essential for the secretion of the protein from the liver cell, but is subsequently cleaved from the amino-terminal end of the polypeptide (Welch, Transferrin: The Iron Carrier: 73-74 (1992)). The present invention provides Tf conjugates comprising either full length as well as mature forms of Tf. The present invention also provides Tf conjugates of recombinant Tf including tagged molecules, for example with a hexa-histadine, GST, TAP, CBP, MBP, FLAG, HA, Myc, Biotin, or any other well known tag in the art. The present invention also provides Tf conjugates of fragments of Tf that retain both iron and TfR binding affinity.

The present invention provides improved Tf conjugates with increased cellular associations. Examples of Tf conjugates which can be improved by incorporation of the present invention, and methods of making such conjugates are well known in the art and can be found, without limitation, in U.S. Pat. Nos. 4,522,750, 4,625,014, 5,000,935, 5,108,987, 5,208,021, 5,208,323, 5,254,342, 5,352,447, 5,354,844, 5,393,737, 5,521,291, 5,547,932, 5,622,929, 5,672,683, 5,728,383, 5,792,458, 5,977,307, 6,315,978, 6,340,701, 6,825,166, 6,878,805, 6,962,686, 7,001,991, and 7,176,278, as well as in Qian et al. (Pharmacol Rev., 54(4):561-87 (2002)), Ogris et al. (Somat Cell Mol Genet., 27(1-6):85-95 (2002)), Weaver and Laske (J Neurooncol., 65(1):3-13 (2003)), Lai et al., (Expert Opin Ther Targets., 9(5):995-1007 (2005)), Gaillard et al., (Expert Opin Drug Deliv., 2(2):299-309 (2005)), and Jones and Shusta (Pharm Res., 24(9):1759-71 (2007)), all of which are hereby incorporated by reference in their entirety for all purposes.

In one embodiment, the present invention provides methods of treating cancer in a subject. In one embodiment, the method comprises administering a Tf conjugate with decreased iron release kinetics, as compared to native Tf conjugates, to a subject with cancer. In another embodiment, the Tf conjugate has increased cellular association or internalization. In one embodiment, the Tf conjugate comprises a cytotoxin or anticancer agent. In another embodiment, Tf is conjugated to a nucleic acid or liposomally encapsulated nucleic acid. In another embodiment, Tf is conjugated to a liposomally encapsulated anti-cancer therapeutic. In one embodiment, the cytotoxin is a diphtheria toxin. In a particular embodiment, the diphtheria toxin is CRM107. In one embodiment, the present invention provides methods of treating cancer in a mammal. In some embodiments, the mammal is a human, mouse, rat, hamster, guinea pig, rabbit, or monkey. Tf conjugates described elsewhere in the application are particularly well suited for use in the methods described here.

The methods of the present invention may be used alone or in combination with adjuvant cancer therapies, including hormone therapy, chemotherapy, radiation therapy, immunotherapy, or surgery.

In one embodiment, the present invention provides formulations and compositions of Tf conjugates with decreased iron release kinetics, increased cellular association, or increased cellular internalization. In one embodiment, the Tf conjugate comprises a cytotoxic agent or anti-cancer agent, for example a diphtheria toxin. In a particular embodiment, the toxin is CRM107. One of skill in the art will know of other suitable anti-cancer agents that may be conjugated to a Tf molecule of the present invention.

The Tf conjugates of the present invention can be administered alone or in mixture with a physiologically acceptable carrier. Such carriers include, but are not limited to, physiological saline or phosphate buffer selected in accordance with the route of administration and standard pharmaceutical practice. Other suitable carriers include, e.g., water, buffered water, saline solutions of from about 0.1% to about 1.0%, glycine solutions of from about 0.1% to about 1.0%, and the like. The compositions of the present invention may additionally contain pharmaceutically acceptable auxiliary substances. Such substances may include pH adjusting agents, buffering agents, tonicity adjusting agents, salts, ion chelators, and the like, for example, sodium acetate, sodium lactate, sodium chloride, potassium chloride, calcium chloride, EDTA, EGTA, carbonate salts, oxalate salts, etc.

In one embodiment, the present invention provides methods of making and screening mutants of Tf with decreased iron release kinetics, increased cellular association, or increased cellular internalization. In one embodiment, the method comprises the steps of mutating a nucleic acid sequence encoding a Tf and then assaying the resultant Tf protein encoded by the mutated nucleic acid for a desired property. In some embodiments, the desired property is decreased iron release kinetics, increased cellular association, or increased cellular internalization. Methods of mutating nucleic acids are well known in the art. Examples of these methods can be found, for example, in CSH Protocols found on the Cold Springs Harbor Protocols website (cshprotocols.org), and in Samsbrook et al., Molecular Cloning: a Laboratory Manual. 3rd edition: CSHL Press, 2001.

In one embodiment, the present invention provides an anti-cancer therapeutic comprising an anti-cancer agent conjugated to a transferrin (Tf) molecule, wherein said Tf molecule has reduced iron release kinetics as compared to wild type Tf. In a particular embodiment, Tf is bound to an anion other than carbonate. In another particular embodiment, the anion is oxalate. In one embodiment of the present invention, said Tf molecule comprises a mutation that results in reduced iron release kinetics. In one embodiment, the amino acid sequence of Tf is 85% identical to the amino acid sequence of SEQ ID NO:1. In another embodiment, said Tf further comprises at least one mutation of a residue selected from the group consisting of K206, K296, H349, H350, K534, R632, D634, and combinations thereof. In one embodiment, the anti-cancer agent conjugated to Tf is a diphtheria toxin. In a particular embodiment of the invention, the diphtheria toxin contains a mutation that reduces non-specific cell-association. In another particular embodiment, the diphtheria toxin is CRM107.

In one embodiment, the present invention provides Tf conjugates with decreased iron release kinetics bound to anions other than carbonate. Anions particularly well suited for use in the present invention include monovalent and divalent anions. Examples of monovalent anions suitable for use in the present invention include without limitation, bromate, chlorate, iodate, nitrate, bisulfate, bisulfate, and the like. Divalent anions well suited for use in the present invention include without limitation, oxalate, hydrogen phosphate, sulfate, malonate, succinate, thiosulfate, sulfite, and the like. One of skill in the art will know of other anions well suited for use in the present invention.

### Structural Information

Transferrins are a group of iron-binding proteins that include serum transferrins, ovotransferrins, and lactoferrins, which share a high degree of structural conservation. The global folds of these proteins consist of two highly similar lobes, the N- and C-lobes, further divided into two domains, commonly referred to as NI, NII, CI, and CII. Both the N- and C-lobes of transferrin bind a single iron atom with high affinity. These Fe³⁺ ions are coordinated by two tyrosines, Y95 and Y188 in the N-lobe and Y426 and Y517 in the C-lobe, a lysine, K206 in the N-lobe and K534 in the C-lobe, a histidine, H249 in the N-lobe and H585 in the C-lobe, and an anion in both lobes, such as carbonate or oxalate. Structural data suggest that residues G65, E83, Y85, R124, K206, S248, and K296 further stabilize the iron atom in the N-lobe (MacGillivray et al, Biochemistry 37:7919-7928 (1998)).

Iron release is a pH-dependent process that occurs in acidic endosomes, where the pH drops from about pH 7.4 in the serum, to about pH 5.6 in the endosome. The mechanisms of iron release are well known and involve protonation of a dilysine trigger, K206 and K296, in the N-lobe and of a triad of residues, K534, R632, and D634, in the C-lobe. Mutation of these pH sensitive motifs is known to cause a decrease in the iron release kinetics of both the N- and C-lobes. In one embodiment, the present invention provides Tf conjugates with mutated N-lobe motifs. In another embodiment, the present invention provides Tf conjugates with mutated C-lobe motifs. In yet another embodiment, the invention provides Tf conjugates with mutated N- and C-lobes.

The molecular details of transferrin iron binding are well detailed in the art. Many high-resolution crystal structures of the various transferrins have been solved, and shed further light on the mechanisms of iron binding and release. For example, the crystal structure of serum Tf, and various mutants thereof, has been solved for the human protein; PDB IDs 1D3K, 1D4N, 1A8E, 1A8F, 1B3E, 1BTJ, 1FQE, 1FQF, 1JQF, 1N84, 1OQG, 1OQH, 2O84, 1BP5, 1DTG, 1N7W, 1N7X, 1RYO, 2HAU, 2HAV, and 2O7U, the rabbit protein; PDB ID 1TFD and 1JNF, the porcine protein; 1H76, and the chicken protein; PDB ID 1RYX and 1N04. Structures are also known for chicken ovotranferrin; PDB ID 1NNT, 1NFT, 1TFA, 2D3I, 1IQ7, 1OVT, and 1IEJ, human lactoferrin; 1VFE, 1VFD, 1B0L, 1FCK, and 1LFG, duck ovotransferrin; 1AOV and 1DOT, camel ovotransferrin; 1DTZ, bovine lactoferrin; 1BLF, equine lactoferrin: 1I6B and 1QJM and camal lactoferrin: 1I6Q. Structural information is also available for the interaction between Tf and TfR, PDB ID 1 SUV (Cheng et al., Cell 166:565-576 (2004)).

One of skill in the art will instantly recognize that due to the high sequence and structural conservation of the different transferrins, biochemistry and structural biology studied for one species will be applicable to the analysis of a second species. Given the breadth of structural and biochemical information available for this family of proteins, one of skill in the art will be able to generate additional mutations in transferrin that reduce the iron release kinetics. Additionally, one of skill in the art will recognize amino acid positions that can be further mutated without consequence to the functional or structural properties of the Tf conjugates taught in the present invention.

### DEFINITIONS

“Cancer” refers without limitation to mammalian cancers, for example, human or marine cancers and carcinomas, sarcomas, adenocarcinomas, lymphomas, leukemias, etc., including solid and lymphoid cancers, brain, kidney, breast, lung, bladder, colon, ovarian, prostate, pancreas, stomach, head and neck, skin, uterine, testicular, glioma, esophagus, and liver cancer, including glioblastoma multiforme tumors, hepatocarcinoma, lymphoma, including B-acute lymphoblastic lymphoma, non-Hodgkin's lymphomas (e.g., Burkitt's, Small Cell, and Large Cell lymphomas) and Hodgkin's lymphoma, leukemia (including AML, ALL, and CML), and multiple myeloma.

“Therapeutic treatment” and “cancer therapies” refer to chemotherapy, hormonal therapy, radiotherapy, and immunotherapy.

As used herein, “treatment” refers to clinical intervention in an attempt to alter the natural course of the individual or cell being treated, for example a cancer cell or tumor in the present invention, and may be performed either for prophylaxis or during the course of clinical pathology. Desirable effects include preventing occurrence or recurrence of disease, reduction of tumor mass, alleviation of symptoms, diminishment of any direct or indirect pathological consequences of the disease, preventing metastasis, lowering the rate of disease progression, amelioration or palliation of the disease state, and remission or improved prognosis.

An “effective amount” or a “therapeutically effective amount” is an amount sufficient to effect a beneficial or desired clinical result, particularly the reduction of a tumor mass, or noticeable improvement in a clinical condition such as cancer. In terms of clinical response for subjects bearing a neoplastic disease, an effective amount is amount sufficient to palliate, ameliorate, stabilize, reverse or slow progression of the disease, or otherwise reduce pathological consequences of the disease. An effective amount may be given in single or divided doses.

The terms “cancer-associated antigen” or “tumor-specific marker” or “tumor marker” interchangeably refers to a molecule (typically protein or nucleic acid such as RNA) that is expressed in the cell, expressed on the surface of a cancer cell or secreted by a cancer cell in comparison to a normal cell, and which is useful for the diagnosis of cancer, for providing a prognosis, and for preferential targeting of a pharmacological agent to the cancer cell. Oftentimes, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. Oftentimes, a cancer-associated antigen is a cell surface molecule that is inappropriately synthesized in the cancer cell, for instance, a molecule that contains deletions, additions or mutations in comparison to the molecule expressed on a normal cell. Oftentimes, a cancer-associated antigen will be expressed exclusively on the cell surface of a cancer cell and not synthesized or expressed on the surface of a normal cell. Exemplified cell surface tumor markers include the proteins tranferrin receptor (TfR) for multiple forms of cancer including brain cancer, c-erbB-2 and human epidermal growth factor receptor (HER) for breast cancer, PSMA for prostate cancer, and carbohydrate mucins in numerous cancers, including breast, ovarian and colorectal.

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100, 200, 300, 400, 500, or more amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of cancer antigens. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. One of skill in the art will also recognize that conservative substitutions to a protein embraced by the present invention will be well tolerated, especially when made in residues not involved in iron binding or TfR association. One of skill in the art will recognize that conservative mutations made in residues involved in iron binding or TfR association may be well tolerated and can be designed by inspection of high resolution structural information readily available in the art.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

“Decreased iron binding kinetics” or “reduced iron binding kinetics” refer to modified or recombinant iron binding proteins or polypeptides, which release bound iron at a slower rate than that of the native or wild type protein. In the context of this application, the modified or recombinant iron binding protein is for example Transferrin, or a functional polypeptide thereof. The reduced iron binding kinetics may be from about 5% to about 100% or more slower for the modified protein, for example 5%, 10%, 20%, 30%, 40%, 50%, 60%, 70%, 80%, 90%, 100%, or more, as compared to the rate of iron release for the native or wild type protein. In certain embodiments, the reduced iron release rate may be from about 1-fold to about 10-fold slower, for example, 1-fold, 2-fold, 3-fold, 4-fold, 5-fold, 6-fold, 7-fold, 8-fold, 9-fold, 10-fold, or more slower as compared to the native of wild type protein. In other embodiments, the reduced iron release kinetics may be from about 101-fold to about 109-fold reduced, for example 101-fold, 102-fold, 103-fold, 104-fold, 105-fold, 106-fold, 107-fold, 108-fold, 109-fold, or more slower as compared to the native or wild type protein. In certain embodiments, the iron release kinetics may be slow enough that they are not readily detectable. In some embodiments, the decreased iron release kinetics may refer to the iron release from the N-lobe, the C-lobe, or both lobes. In other embodiments, the iron release kinetics may refer to the iron release rate at a single pH, for example at a physiologically relevant pH from about 5.0 to about 8.0, for example, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In other embodiments, the iron release kinetics may be reduced at multiple pH.

As used herein, the term “salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the basic compounds of the present invention are salts formed with acids, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

### EXAMPLES

### Example 1

The following example illustrates the use of a mathematical model for Transferrin/Transferrin Receptor trafficking for the identification of kinetic properties that can be altered to increase therapeutic value.

To investigate whether Tf itself could be modified to change its cellular trafficking behavior, we employed a mathematical model of Tf/TfR trafficking. The use of models for the study of the Tf/TfR trafficking system is well-established, though typically such models are used to analyze and better understand experimental data (Yazdi and Murphy, Cancer Res. 1994; 54(24):6387-94; Ciechanover et al., J Biol Chem. 1983; 258(16):9681-9). Here, we have used the model as a convenient framework to allow a mathematical assessment of how changes in intracellular trafficking could result from alteration of Tf properties. By systematically varying the values of seven different Tf parameters, the model indicated that decreasing the rate of iron release from Tf is a potential strategy for increasing cellular association. This design criterion is novel, since it alters the ligand/metal interaction, instead of the ligand/receptor interaction, for the engineering of protein-based drug delivery vehicles.

A previously reported Tf/TfR trafficking model was integrated with a model of endosomal sorting (French and Lauffenburger, Ann Biomed Eng. 1997; 25(4):690-707; Ciechanover et al., J Biol Chem. 1983; 258(16):9681-9). When necessary, additional terms were added to enable the trafficking simulation of Tf ligands with modified properties. For example, to enable the simulation of an iron-free Tf (apo-Tf) ligand with an increased association rate for TfR, a term describing the association of apo-Tf for TfR was added to the species balance of the apo-Tf/TfR surface complex. This was not accounted for in the original model, since the apo-form of native Tf does not bind TfR. A full list of model equations and parameters is provided here.

Model Equations

Bulk & Surface Equations. Species balance for bulk extracellular FeTf

$$\frac{d ( {FeTf}_{bulk} )}{d t} = ( \begin{matrix}- {k}_{{F}_{e} Tf , TfR} {FeTf}_{bulk} {TfR}_{surf} + \\ {k}_{{F}_{e} Tf , TfR , r} {FeTf\_TfR}_{surf} + \\ {k}_{rec} {FeTf}_{rec}\end{matrix} ) \frac{{n}_{cell}}{{V}_{bulk} {N}_{A}}$$

Species balance for bulk extracellular Tf

$$\frac{d ( {Tf}_{bulk} )}{d t} = ( \begin{matrix}\begin{matrix}- {k}_{Tf , TfR} {Tf}_{bulk} {TfR}_{surf} + \\ {k}_{Tf , TfR , r} {Tf\_TfR}_{surf} +\end{matrix} \\ {k}_{rec} {Tf}_{rec}\end{matrix} ) \frac{{n}_{cell}}{{V}_{bulk} {N}_{A}}$$

Species balance for surface TfR

$$\frac{d ( {TfR}_{surf} )}{d t} = ( \begin{matrix}\begin{matrix}\begin{matrix}- {k}_{{F}_{e} Tf , TfR} {FeTf}_{bulk} {TfR}_{surf} - \\ {k}_{Tf , TfR} {Tf}_{bulk} {TfR}_{surf} + \\ {k}_{{F}_{e} Tf , TfR , r} {FeTf\_TfR}_{surf} + \\ {k}_{Tf , TfR , r} {Tf\_TfR}_{surf} - \\ {k}_{int} {TfR}_{surf} + \\ {k}_{rec} {TfR}_{rec} +\end{matrix} \\ k deg {TfR}_{deg} +\end{matrix} \\ {k}_{deg} {FeTf\_TfR}_{deg} + \\ {k}_{deg} {Tf\_TfR}_{deg}\end{matrix} ) \frac{{n}_{cell}}{{V}_{bulk } {N}_{A}}$$

Species balance for surface FeTf/TfR complex

$$\frac{d ( {FeTf\_TfR}_{surf} )}{d t} = + {k}_{FeTf , TfR} {FeTf}_{bulk} {TfR}_{surf} - {k}_{FeTf , TfR , r} {FeTf\_TfR}_{surf} - {k}_{int} {FeTf\_TfR}_{surf} + {k}_{rec} {FeTf\_TfR}_{rec}$$

Species balance for surface Tf/TfR complex

$$\frac{d ( {Tf\_TfR}_{surf} )}{d t} = + {k}_{Tf , TfR} {Tf}_{bulk} {TfR}_{surf} - {k}_{Tf , TfR , r} {Tf\_TfR}_{surf} - {k}_{int} {Tf\_TfR}_{surf} + {k}_{rec} {Tf\_TfR}_{rec}$$

Vesicular Equations

Species balance for vesicular FeTf

$$\frac{d ( {FeTf}_{ves} )}{d t} = - {k}_{Fe , rel} {FeTf}_{ves} - {k}_{endo} {FeTf}_{ves} {TfR}_{ves} + {k}_{endo , r} {FeTf\_TfR}_{ves} - {k}_{endo} {FeTf}_{ves} {TfR}_{tub} + ( 1 + κ ) {k}_{endo , r} {FeTf\_TfR}_{tub} - {k}_{s υ} {FeTf}_{ves} - {k}_{st} {FeTf}_{ves}$$

Species balance for vesicular Tf

$$\frac{d ( {Tf}_{ves} )}{d t} = + {k}_{Fe , rel} {FeTf}_{ves} - {k}_{endo} {Tf}_{ves} {TfR}_{ves} + {k}_{endo , r} {Tf\_TfR}_{ves} - {k}_{endo} {Tf}_{ves } {TfR}_{tub} + ( 1 / κ ) {k}_{endo , r} {Tf\_TfR}_{tub} - {k}_{s υ} {Tf}_{ves} - {k}_{st} {Tf}_{ves}$$

Species balance for vesicular TfR

$$\frac{d ( {TfR}_{ves} )}{d t} = + {k}_{int} {TfR}_{surf} - {k}_{endo} {FeTf}_{ves} {TfR}_{ves} - {k}_{endo} {Tf}_{ves} {TfR}_{ves} + {k}_{endo , r} {FeTf\_TfR}_{ves} + {k}_{endo , r} {Tf\_TfR}_{ves} - γ {TfR}_{ves} - {k}_{s υ} {TfR}_{ves}$$

Species balance for vesicular FeTf/TfR complex

$$\frac{d ( {FeTf\_TfR}_{ves} )}{d t} = - {k}_{Fe , rel} {FeTf\_TfR}_{ves} - {k}_{endo , r} {FeTf\_TfR}_{ves} + {k}_{endo} {FeTf}_{ves} {TfR}_{ves} + {k}_{int} {FeTf\_TfR}_{surf} - γ {FeTf\_TfR}_{ves} - {k}_{s υ} {FeTf\_TfR}_{ves}$$

Species balance for vesicular Tf/TfR complex

$$\frac{d ( {Tf\_TfR}_{ves}}{d t} = + {k}_{Fe , rel} {FeTf\_TfR}_{ves} - {k}_{endo , r} {Tf\_TfR}_{ves} + {k}_{endo} {Tf}_{ves} {TfR}_{ves} - {γTf\_TfR}_{ves} - {k}_{sv} {Tf\_TfR}_{ves} + {k}_{int} {Tf\_TfR}_{surf}$$

Tubular Equations

Species balance for tubular FeTf/TfR complex

$$\frac{d ( {FeTf\_TfR}_{tub} )}{d t} = - {k}_{Fe , rel} {FeTf\_TfR}_{tub} - {k}_{endo , r} {FeTf\_TfR}_{tub} + κ {k}_{endo} {FeTf}_{ves} {TfR}_{tub} + γ {FeTf\_TfR}_{ves} - {k}_{st} {FeTf\_TfR}_{tub}$$

Species balance for tubular Tf/TfR complex

$$\frac{d ( {Tf\_TfR}_{tub} )}{d t} = + {k}_{Fe , rel} {FeTf\_TfR}_{tub} - {k}_{endo , r} {Tf\_TfR}_{tub} + κ {k}_{endo} {Tf}_{ves} {TfR}_{tub} + {γTf\_TfR}_{ves} - {k}_{st} {Tf\_TfR}_{tub}$$

Species balance for tubular TfR

$$\frac{d ( {TfR}_{tub } )}{d t} = - κ {k}_{endo} {FeTf}_{ves} {TfR}_{tub} - κ {k}_{endo} {Tf}_{ves} {TfR}_{tub} + {k}_{endo , r} {FeTf\_TfR}_{tub} + {k}_{endo , r} {Tf\_TfR}_{tub} + γ {TfR}_{ves} - {k}_{st} {TfR}_{tub}$$

Recycling Equations

Species balance for recycled FeTf

$$\frac{d ( {FeTf}_{rec} )}{d t} = + {k}_{st} {FeTf}_{ves} - {k}_{rec} {FeTf}_{rec}$$

Species balance for recycled Tf

$$\frac{d ( {Tf}_{rec} )}{d t} = + {k}_{st} {Tf}_{ves} - {k}_{rec} {Tf}_{rec}$$

Species balance for recycled TfR

$$\frac{d ( {TfR}_{rec} )}{d t} + {k}_{st} {TfR}_{tub} - {k}_{rec} {TfR}_{rec}$$

Species balance for recycled FeTf/TfR complex

$$\frac{d ( {FeTf\_TfR}_{rec} )}{d t} = + {k}_{st} {FeTf\_TfR}_{tub} - {k}_{rec} {FeTf\_TfR}_{rec}$$

Species balance for recycled Tf/TfR complex

$$\frac{d ( {Tf\_TfR}_{rec} )}{d t} = + {k}_{st} {Tf\_TfR}_{tub} - {k}_{rec} {Tf\_TfR}_{rec}$$

Degradation Equations

Species balance for degraded FeTf

$$\frac{d ( {FeTf}_{deg} )}{d t} = + {k}_{sv} {FeTf}_{ves} - {k}_{deg} {FeTf}_{deg}$$

Species balance for degraded Tf

$$\frac{d ( {Tf}_{deg} )}{d t} = + {k}_{sv} {Tf}_{ves} - {k}_{deg} {Tf}_{deg}$$

Species balance for degraded TfR

$$\frac{d ( {TfR}_{deg} )}{d t} = + {k}_{sv} {TfR}_{ves} - {k}_{deg} {TfR}_{deg}$$

Species balance for degraded FeTf/TfR complex

$$\frac{d ( {FeTf\_TfR}_{deg} )}{d t} = + {k}_{sv} {FeTf\_TfR}_{ves} - {k}_{deg} {FeTf\_TfR}_{deg}$$

Species balance for degraded Tf/TfR complex

$$\frac{d ( {Tf\_TfR}_{deg} )}{d t} = + {k}_{sv} {Tf\_TfR}_{ves} - {k}_{deg} {Tf\_TfR}_{deg}$$

Internalized Tf Equation

Species balance for total internalized Tf

$$Internalized = ( 1 + κ ) {FeTf}_{ves} + ( 1 + κ ) {Tf}_{ves} + {FeTf\_TfR}_{ves} + {Tf\_TfR}_{ves} + {FeTf\_TfR}_{tub} + {Tf\_TfR}_{tub} + {FeTf}_{rec} + {Tf}_{rec} + {FeTf\_TfR}_{rec} + {Tf\_TfR}_{rec} + {FeTf}_{deg} + {Tf}_{deg } + {FeTf\_TfR}_{deg} + {Tf\_TfR}_{deg}$$

Model equations were solved with initial conditions of zero for all species except the concentration of iron-loaded diferric Tf, or holo-Tf, in the media (1 nM) and the number of transferrin receptors on the cell surface (5.4×10⁵ receptors) (Yazdi and Murphy, Cancer Res. 1994; 54(24):6387-94). The length of the simulations was 50 h. To quantify cellular association of Tf ligands, the area under the curve (AUC) of internalized Tf vs. time was calculated.

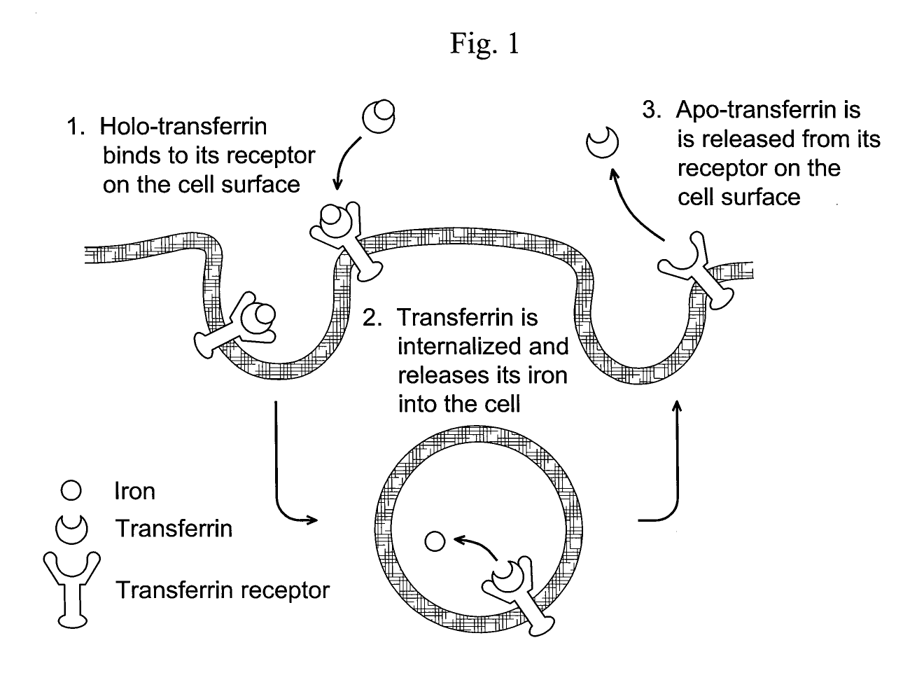

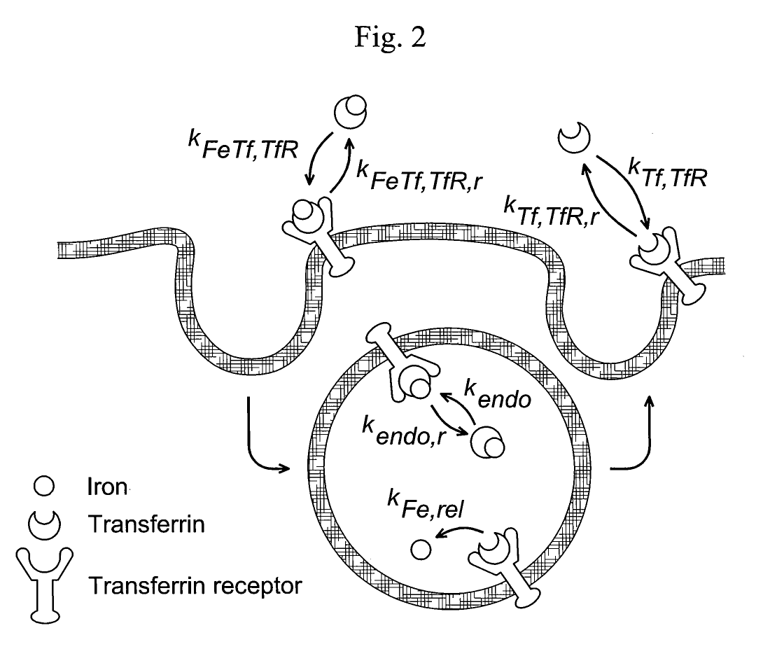

Mathematical modeling identifies Tf iron release as a factor governing cellular association. The transport of Tf into cells has been extremely well characterized and serves as a model for receptor mediated endocytosis (Hentze and Muckenthaler, Cell. 2004; 117(3):285-97) (FIG. 1). To determine how Tf can be modified to increase cellular association, we developed a mathematical model of the Tf/TfR trafficking pathway to assess how changes in properties of Tf might affect its trafficking through cells. We considered seven different properties associated with ligand/receptor and ligand/metal interactions that could in principle be modified (Table 4). The specific properties in the context of the Tf/TfR trafficking cycle are shown in FIG. 2.

TABLE 4
List of potentially modifiable Tf molecular properties

| Rate | Definition | Native Value | Reference |
|---|---|---|---|
| k_{FeTf, TfR}^{†} | Association | 4 ± 1 × 10⁷ | Yazdi and Murphy, |
| | rate of holo- | M⁻¹ min⁻¹ | Cancer Res. |
| | Tf for TfR | | 1994; 54(24): 6387-94 |
| k_{FeTf, TfR, r}^{†} | Dissociation | 1.3 ± 0.5 min⁻¹ | Yazdi and Murphy, |
| | rate of holo- | | Cancer Res. |
| | Tf from TfR | | 1994; 54(24): 6387-94 |
| k_{Tf, TfR} | Association | 0.0 M⁻¹ min⁻¹ | Lebron et al., Cell. |
| | rate of apo- | | 1998; 93(1): 111-23 |
| | Tf for TfR | | |
| k_{Tf, TfR, r} | Dissociation | 2.6 min⁻¹ | Ciechanover et al., |
| | rate of apo- | | J Biol Chem. 1983; |
| | Tf from TfR | | 258(16): 9681-9 |
| k_{endo}^{‡} | Endosomal | 4.4 ± 0.4 × 10⁷ | Lebron et al., Cell. |
| | association | M⁻¹ min⁻¹ | 1998; 93(1): 111-23 |
| | rate of Tf | | |
| | for TfR | | |
| k_{endo, r}^{‡} | Endosomal | 0.056 ± 0.012 min⁻¹ | Lebron et al., Cell. |
| | dissociation | | 1998; 93(1): 111-23 |
| | rate of Tf | | |
| | from TfR | | |
| k_{Fe, rel} | Tf iron | 1.0 × 10² min⁻¹ | Est. |
| | release rate | | |

^{†}Range reported is the 95% confidence interval.
^{‡}Range reported is the mean and standard deviation.

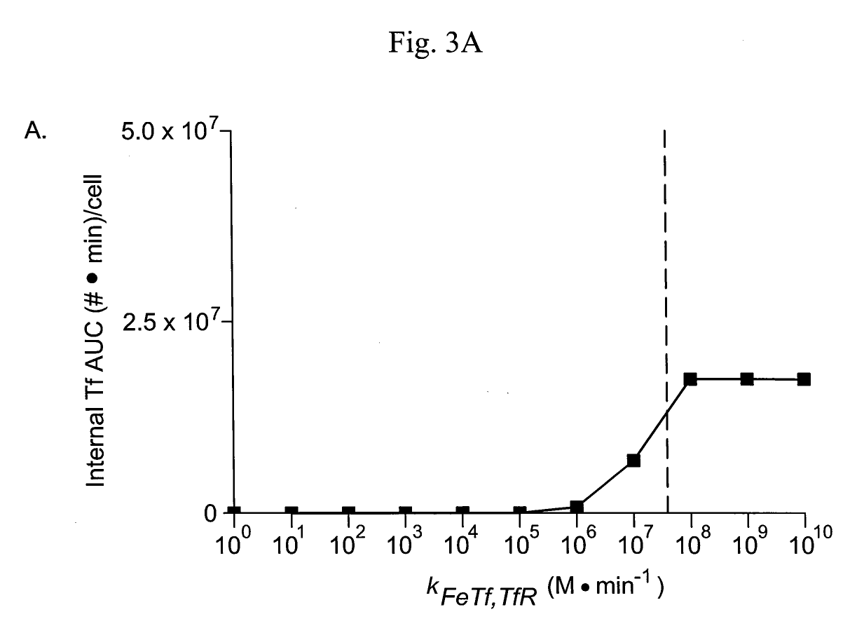

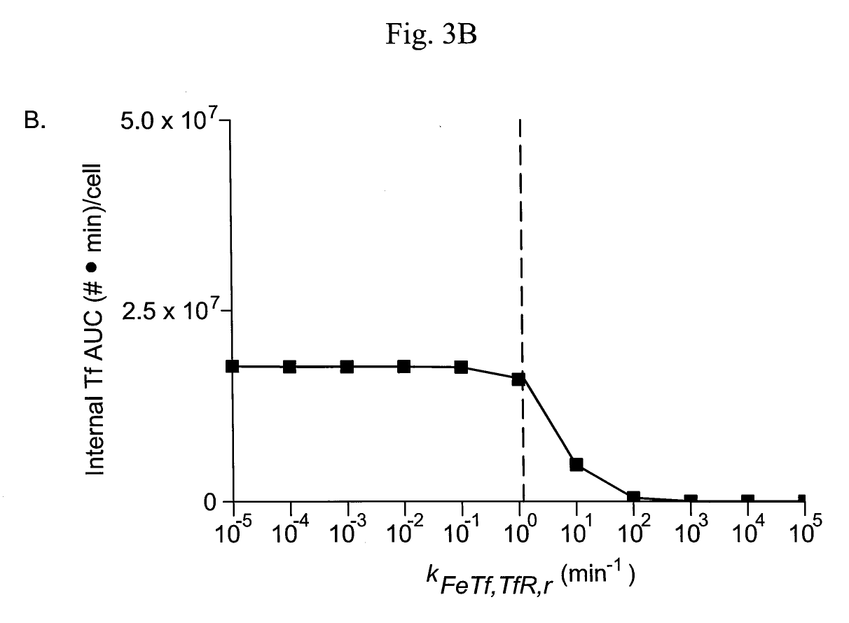

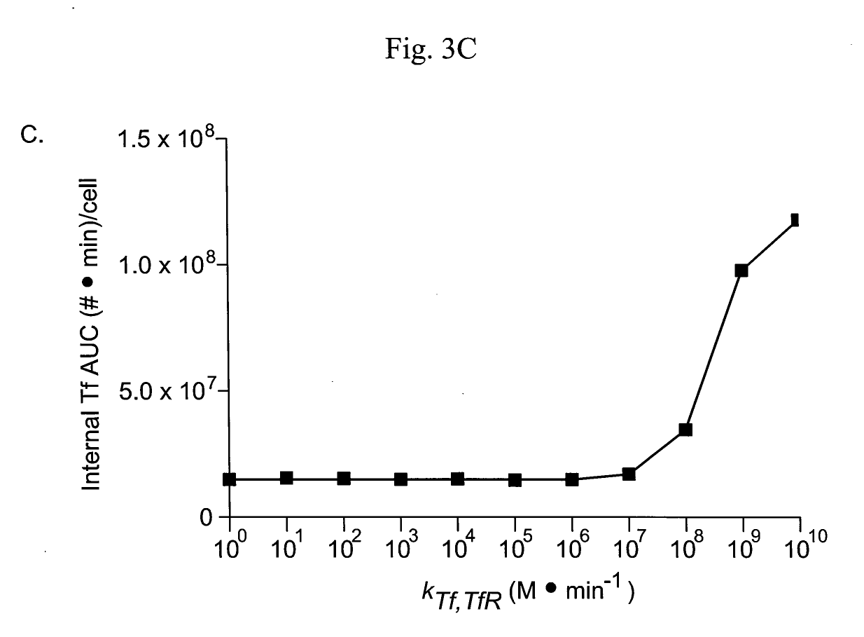

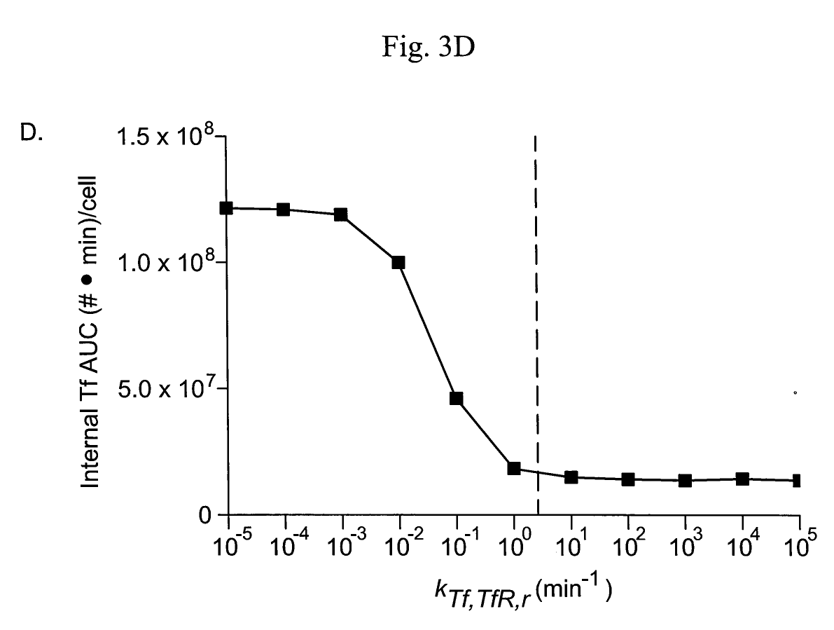

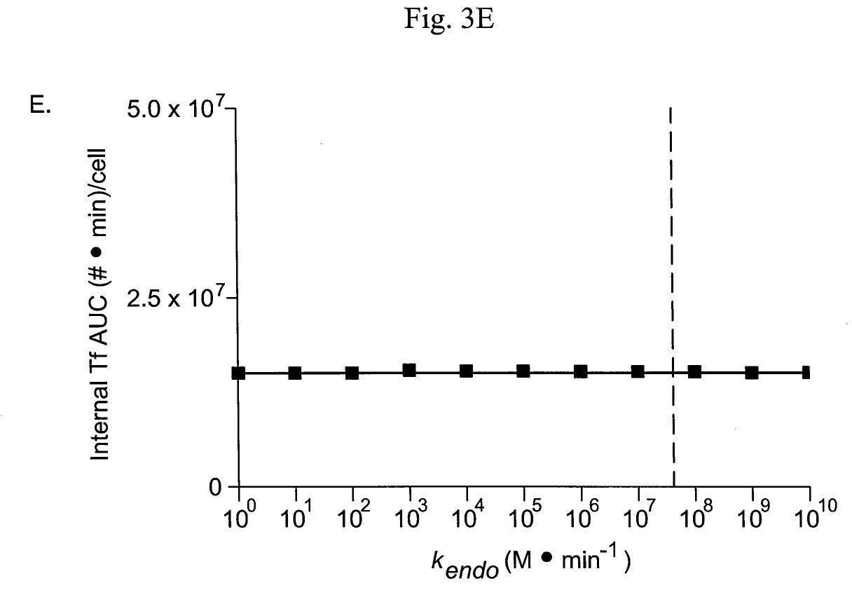

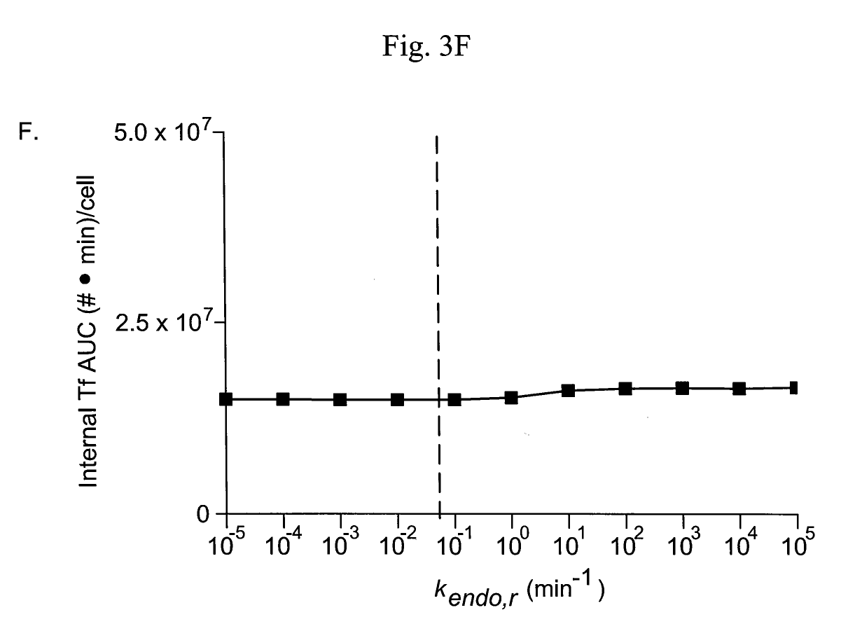

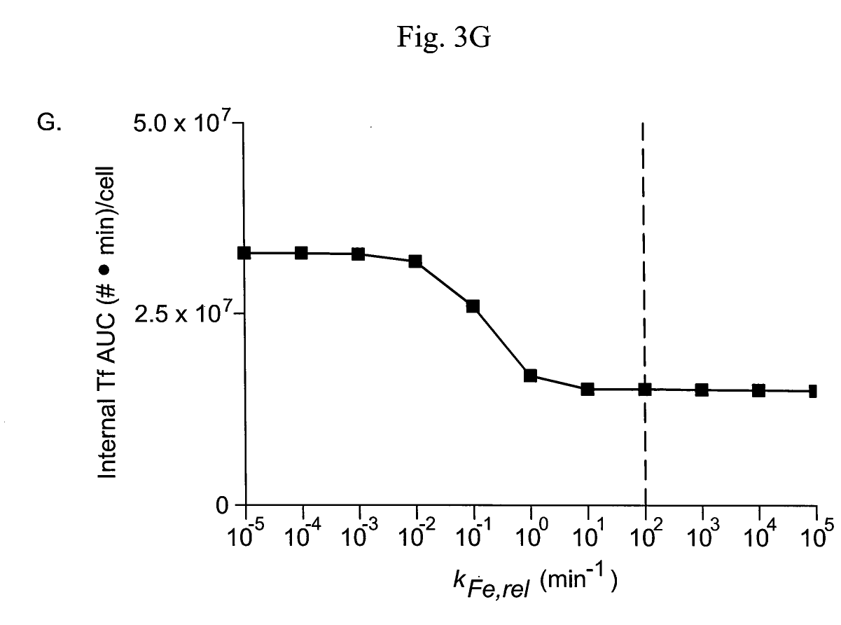

The effects of modifying these properties on the degree of cellular association were investigated by ranging the value of each property over several orders of magnitude (FIG. 3). Cellular association is quantified by taking the area under the curve (AUC) of the internalized Tf values generated by model simulations, such that higher AUC values correspond to increased cellular association.

Surprisingly, only minor increases in cellular association are predicted for increasing the affinity of holo-Tf for TfR (FIGS. 3A and 3B). This is due to the fast rate of iron release (2-3 min) from Tf upon internalization into the endosome, which converts holo-Tf into apo-Tf (Bali et al., Biochemistry. 1991; 30(2):324-8). This rapid conversion to apo-Tf from holo-Tf counteracts the expected increase in cellular association from increasing the affinity of holo-Tf for TfR, since apo-Tf has undetectable binding to TfR at neutral pH and is thus quickly released from TfR upon recycling back to the cell surface (Lebron et al., Cell. 1998; 93(1):111-23). This phenomenon accounts for the large increases in cellular association predicted when the binding affinity of apo-Tf for TfR is increased (FIGS. 3C and 3D).

Variation of endosomal binding affinity for TfR was not predicted to lead to a significant change in cellular association. In the endosomal sorting model used, dissociation of Tf from TfR in the endosome raises the likelihood of Tf being routed to the lysosome and degraded. This increases the cellular association of Tf slightly, since the rate constants characterizing the length of time spent in the degradation pathway are lower than those for the recycling pathway. Nevertheless, this increase in cellular association is minimal compared to the predicted increases seen in FIGS. 3C, 3D, and 3G.

FIG. 3G shows that lowering the rate of iron release from Tf is predicted to lead to an increase in Tf cellular association. In this instance, the lowered iron release rate allows Tf to remain as holo-Tf upon being recycled, thus retaining its affinity for TfR.

Several assumptions were used to define the behavior of the model, and are described below.

i. Total Tf receptor number is assumed to be constant. As receptors are degraded, they are replaced through the production of an equal number of new receptors.

ii. Internalization rate is assumed to be the same for free receptors as for bound receptors.

iii. Tf/TfR complexes with inhibited iron release are assumed to be recycled to the cell surface like native Tf/TfR complexes, as opposed to being degraded. This is supported by studies on the effects of lysosomotropic agents on the Tf/TfR cycle. Lysosomotropic agents raise the pH level in the endosome, and hence slow the rate of iron release from Tf. The administration of such agents was not found to inhibit the recycling of Tf to the cell surface (Ciechanover et al., J Biol Chem. 1983; 258(16):9681-9).

iv. Direct measurements of the iron release rate within cellular endosomes, k_{Fe,rel}, are unavailable. However, it is known that iron is completely released from Tf that has been internalized into the endosome before Tf is recycled to the cell surface (Ciechanover et al., J Biol Chem. 1983; 258(16):9681-9). Therefore, an iron release rate of 100 min⁻¹ was assumed in the model, which allows all iron from native Tf to be released prior to it being recycled to the cell surface.

v. For the endosomal sorting component of the model, Tf/TfR complexes are assumed to not associate with endosomal retention complexes which promote the degradation of ligand/receptor complexes (Anthony and French, Biotechnology and Bioengineering. 1996; 51(3):281-297). This is consistent with the observation that native Tf is almost completely recycled.

vi. The partition coefficient, K, was calculated according to the expression κ=(1−λ)², where λ is the diameter of Tf divided by the diameter of the tubule (Anthony and French, Biotechnology and Bioengineering. 1996; 51(3):281-297). An average Tf diameter of 60 nM was estimated from the crystal structure of Tf (Cheng et al., Cell. 2004; 116(4):565-76), and a tubule diameter of 600 nM was assumed (Marsh et al., Proc Natl Acad Sci USA. 1986; 83(9):2899-903).

Sensitivity Analysis of Parameter Uncertainty

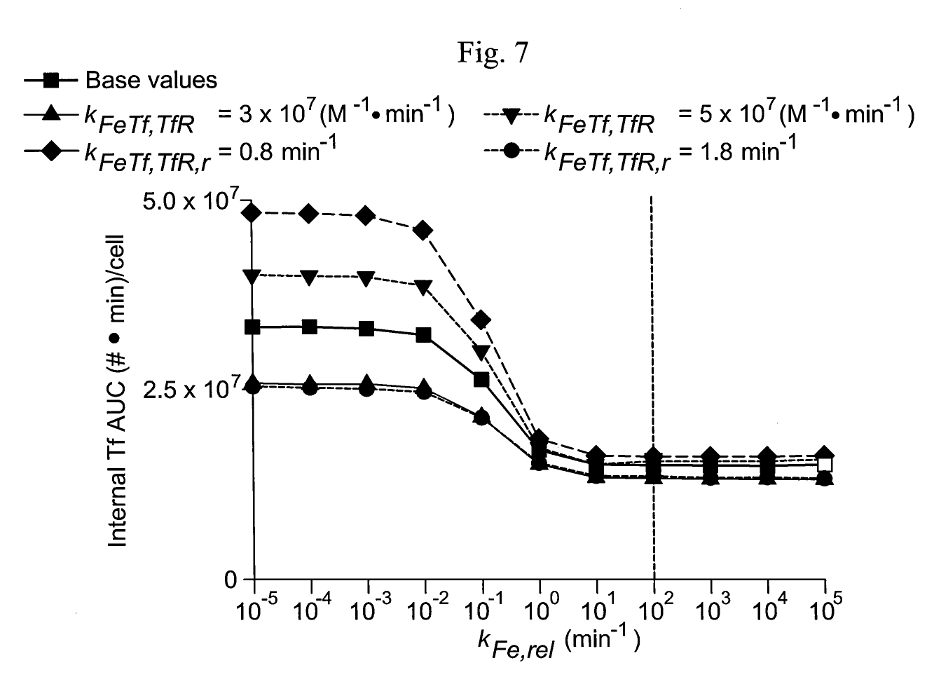

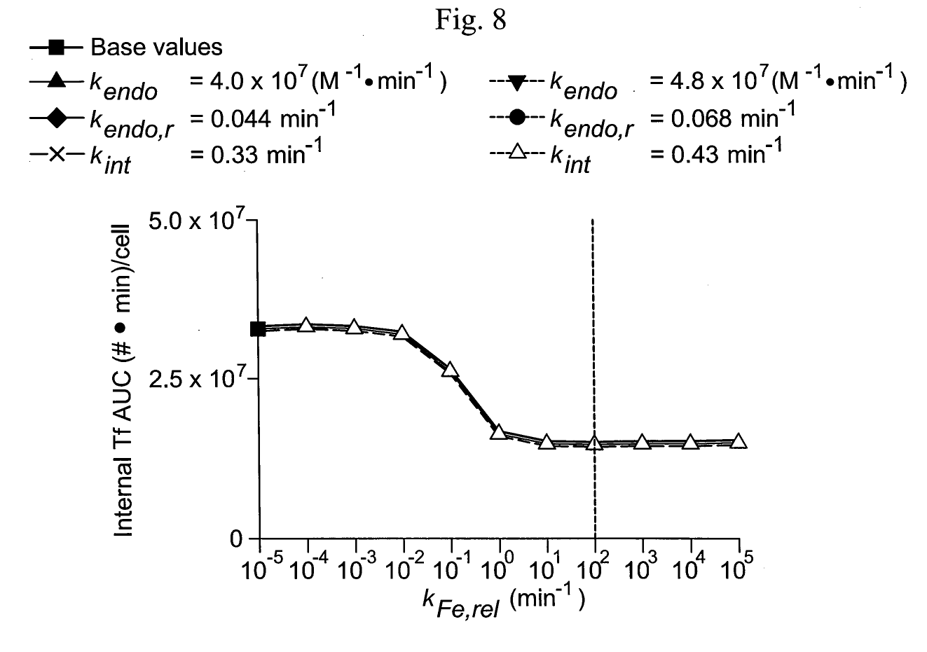

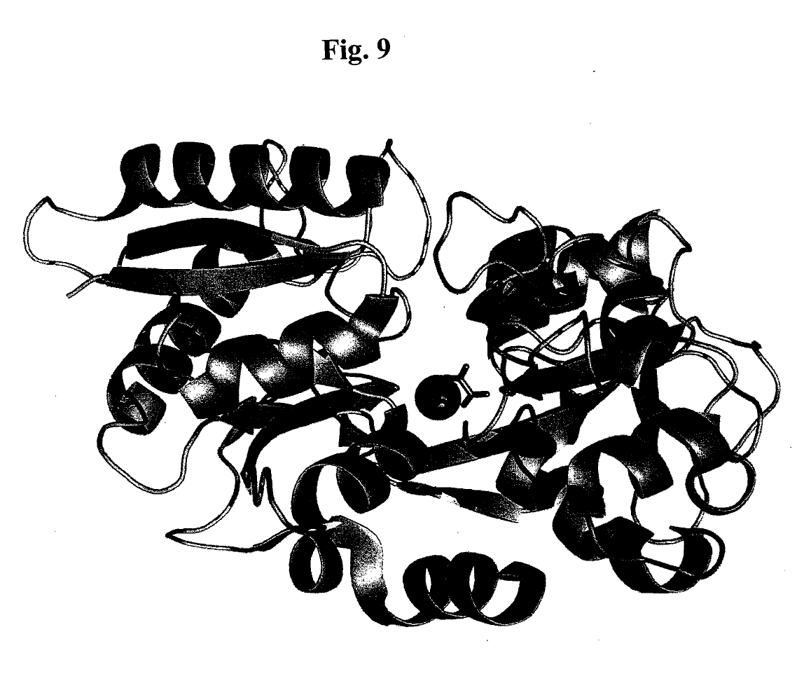

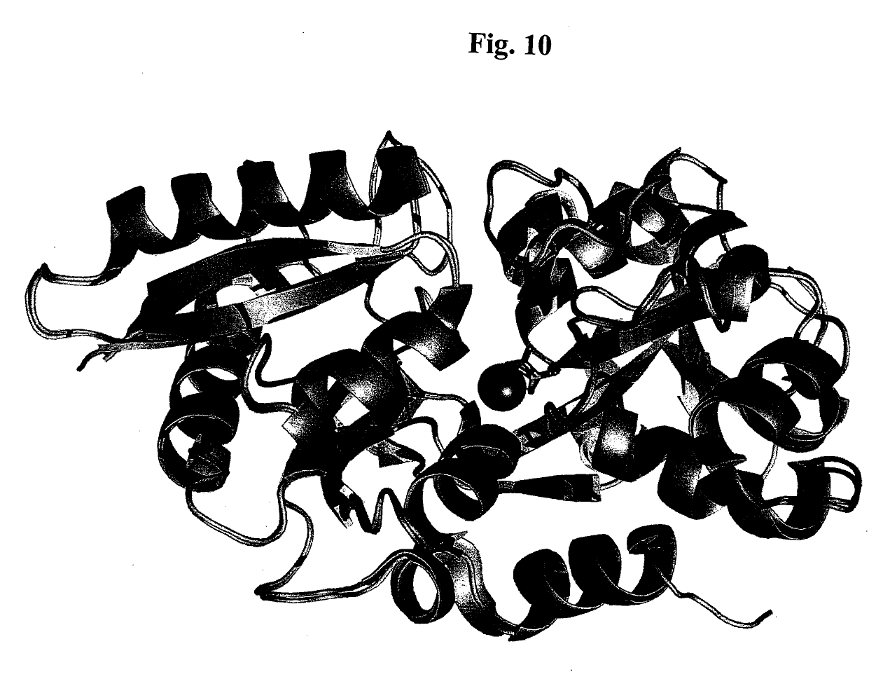

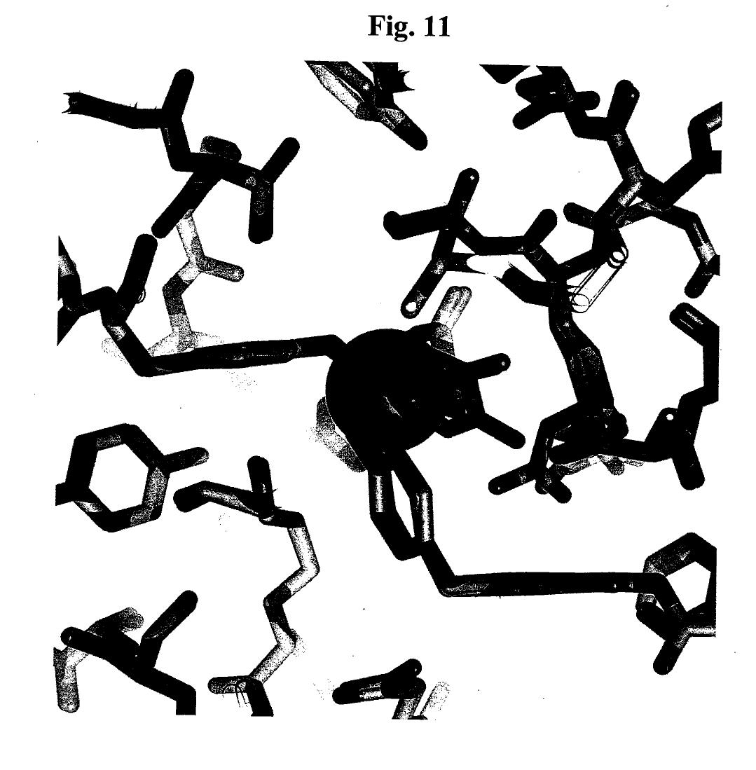

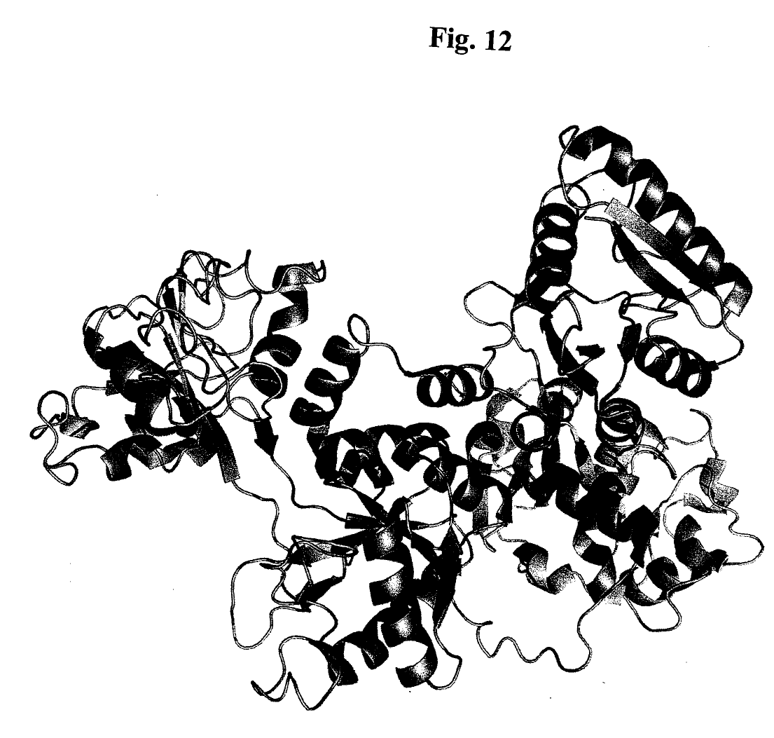

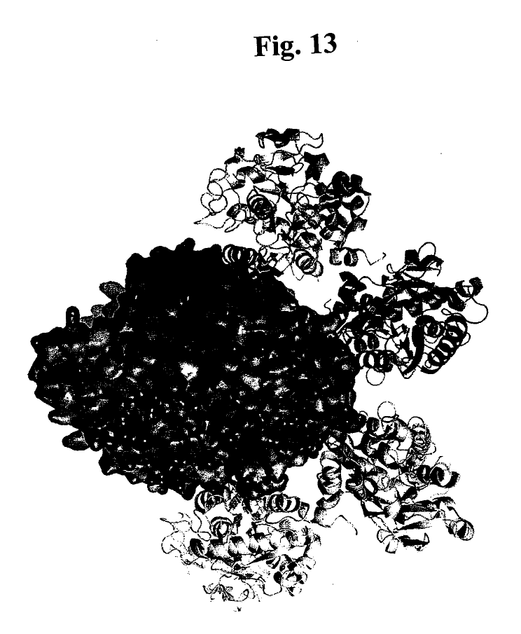

A sensitivity analysis was performed to assess the impact of parameter uncertainty on the prediction that lowering the Tf iron release rate increases cellular association. The AUC values in FIG. 3G were recalculated while setting the value for a particular variable to the lower or upper range of its error interval. The AUC values show the most sensitivity to variations across the error intervals of k_{FeTf,TfR} and k_{FeTf,TfR,r} (FIG. 7), and are relatively insensitive to variations across the error intervals of k_{endo}, k_{endo,r}, and kᵢₙₜ (FIG. 8). In each case where a parameter was varied, lowering the iron release rate was still predicted to increase cellular association.

### Example 2

This Example demonstrates that the reduced iron release kinetics of oxalate Tf result in increased cellular association of oxalate Tf compared to native Tf.

To test the prediction of the model, we generated a version of Tf in which iron release is inhibited. This strategy seemed preferable over attempting to increase the affinity of apo-Tf for TfR, as increasing protein affinity is very challenging (Rao et al., Nat Biotechnol. 2005; 23(2):191-4) whereas the literature contains numerous examples of successful efforts to slow or inhibit iron release from Tf. We chose to replace the synergistic carbonate anion with oxalate, which greatly reduces the iron release rate of iron from Tf without significantly affecting its binding affinity for TfR (Halbrooks et al., J Mol Biol. 2004; 339(1):217-26). A summary of the iron release rates for native Tf and oxalate Tf (Ciechanover et al., J Biol Chem. 1983; 258(16):9681-9) is presented in Table 5.

TABLE 5
Iron release rates of native Tf vs. oxalate Tf in the
presence of 12 mM Tiron (pH 7.4) or 4 mM EDTA (pH 5.6)
(Halbrooks et al., J Mol Biol. 2004; 339(1): 217-26).

| | Native Tf (min⁻¹) | Oxalate Tf (min⁻¹) |
|---|---|---|
| Tf (pH 7.4, N-lobe) | 0.044 ± 0.002 | 0.0005 ± 0.00003 |
| Tf (pH 7.4, C-lobe) | 0.037 ± 0.002 | 0.0032 ± 0.0002 |
| Tf (pH 5.6, N-lobe) | 16.8 ± 1.3 | 0.192 ± 0.008 |
| Tf (pH 5.6, C-lobe) | 0.139 ± 0.006 | 0.0083 ± 0.0010 |

HeLa cells (American Type Culture Collection, Manassas, Va.) were seeded on 35 mm dishes (Becton Dickinson and Company, Franklin Lakes, N.J.) in MEM (Invitrogen, Carlsbad, Calif.) supplemented with 2.2 g/L sodium bicarbonate, 10% FBS (Hyclone, Logan, Utah), 1% sodium pyruvate (Invitrogen), 100 units/mL penicillin (Invitrogen), and 100 μg/mL streptomycin (Invitrogen) at a pH of 7.4. Cells were incubated overnight at 37° C. in a humidified atmosphere with 5% CO₂ to a final density of 4×10⁵ cells/cm². All reagents and materials were purchased from Sigma-Aldrich (St. Louis, Mo.) unless otherwise noted.

Holo-Tf proteins were iodinated with Na¹²⁵I from MP Biomedicals (Irvine, Calif.) using IODO-BEADS from Pierce Biotechnology (Rockford, Ill.). Radiolabeled Tf was purified using a Sephadex G-10 column with bovine serum albumin present to block non-specific binding.

Trafficking experiments were performed in triplicate. After aspirating the seeding medium, incubation medium containing varying concentrations of radiolabeled holo-Tf was added to each dish. This medium was comprised of MEM supplemented with 20 mM HEPES, pH 7.4 containing 1% sodium pyruvate, 100 units/mL penicillin, and 100 μg/mL streptomycin. After 5, 15, 30, 60, 90, or 120 min, the incubation medium was aspirated and the dishes were washed five times with ice-cold WHIPS (20 mM HEPES, pH 7.4 containing 1 mg/mL PVP, 130 mM NaCl, 5 mM KCl, 0.5 mM MgCl₂, and 1 mM CaCl₂) to remove non-specifically bound Tf. Ice-cold acid strip solution (1 mL of 50 mM glycine-HCl, pH 3.0 containing 100 mM NaCl, 1 mg/mL polyvinylpyrrolidone (PVP), and 2 M urea) was then added to each dish. Dishes were placed on ice for 8 min and then washed again with an additional mL of the acid strip solution. Following the removal of the specifically bound Tf on the cell surface by the acid strip washes, NaOH (1 mL of 1 M) was added to the dishes for 30 min to solubilize the cells. After addition of another mL of NaOH, the two basic washes were collected and counted to determine the amount of internalized Tf.

To test our prediction that a lowered iron release rate would increase the cellular association of Tf, we conducted in vitro cell trafficking experiments with HeLa cells. Native Tf and oxalate Tf were radiolabeled with iodine-125 and incubated with HeLa cells for two hours, and the amount of internalized Tf as a function of concentration and time was monitored (FIG. 4). The results show that HeLa cells internalize a significantly greater amount of oxalate Tf compared to native Tf at concentrations of 0.1 nM and 1 nM. At the end of the two hour period, at a concentration of 0.1 nM, the level of native Tf had reached 3.2×10⁴ internalized Tf molecules per cell, compared to 5.0×10⁴ internalized Tf molecules per cell for oxalate Tf (FIG. 4A). Taking the AUC for each curve, we obtain values of 3.1×10⁶ (#·min)/cell for native Tf and 4.5×10⁶ (#·min)/cell for oxalate Tf, an increase of 45%. Similarly, at 1 mM, native Tf had reached 2.4×10⁵ internalized Tf molecules per cell compared to 4.1×10⁵ internalized Tf molecules per cell for oxalate Tf (FIG. 4B). The AUC values of each curve are 2.3×10⁷ (#·min)/cell for native Tf vs. 3.6×10⁷ (#·min)/cell for oxalate Tf, an increase of 57%. At 10 nM, the difference in cellular association between oxalate Tf and native Tf diminishes because the receptors become saturated (FIG. 4C). These results support the hypothesis that inhibition of iron release from Tf can increase its cellular association.

### Example 3

Diphtheria toxin conjugates of oxalate Tf are more cytotoxic against HeLa cells than native Tf conjugates.

DT conjugates of holo-Tf were made using 2-iminothiolane and sulfo-SMCC (Pierce Biotechnology) as crosslinkers. To thiolate DT, 6.4 μL of an iminothiolane-HCl solution (prepared by dissolving 0.5 mg of iminothiolane-HCl in 800 μL of the borate buffer) was added to 0.3 mg of DT dissolved in 60 μL of borate buffer (0.1 M sodium borate, pH 8 containing 1 μM EDTA, 0.15 M NaCl). After 90 minutes at room temperature, the thiolated DT was separated from free iminothiolane by size exclusion chromatography using small spin columns (Zeba Desalt Spin Column, Pierce Biotechnology).

While DT was being thiolated, Tf was reacted with sulfo-SMCC by first dissolving 0.5 mg of sulfo-SMCC in 20 μL of DMSO and then adding that solution to 80 μL of borate buffer. Tf (16 mg) was dissolved in 2 mL of borate buffer and 36 μL of the SMCC solution was added. After 90 minutes at room temperature, the modified Tf-SMCC compound was then separated from free sulfo-SMCC by size exclusion chromatography using the spin columns.

The two modified protein solutions were diluted, incubated together overnight at 4° C., and reduced in volume using centrifugal concentrators (Centriprep YM-10, Millipore, Billerica, Mass.). The DT conjugates with a 1:1 Tf:DT ratio in the concentrated solution were then purified by HPLC (AKTA FPLC Chromato graphic System, GE Healthcare Bio-Sciences, Piscataway, N.J.) using a HiPrep 16/60 Sephacryl S-200 HR size exclusion column (GE Healthcare Bio-Sciences); the identity of each peak was confirmed by SDS-PAGE. The concentration of the 1:1 Tf:DT conjugates was quantified using the absorbance of holo-Tf at 465 nm with a measured extinction coefficient of 0.0506 mL mg⁻¹ cm⁻¹.

The MTT cell proliferation assay was used to determine cell survival according to instructions supplied by the manufacturer (Chemicon International, Temecula, Calif.). Briefly, HeLa cells (2×10⁴ cells) were seeded into each well of a 24-well tissue culture plate with four wells for each condition. After incubating the cells overnight, the seeding medium was aspirated, and the cells were washed with PBS and incubated for 48 hours with 450 μL of fresh seeding medium containing varying concentrations of DT conjugates. Reagent AB (40 μL of 5 mg/mL MTT in PBS) was then added to each well for 4 hours, followed by the addition of 450 μL of Reagent C (isopropanol with 0.04 M HCl) for color development. Visible light absorbance of each well was measured at 570 nm and 630 nm with a plate reader. The survival of cells relative to the control (cells incubated with media containing no DT conjugates) was calculated by taking the ratio of the (A₅₇₀-A₆₃₀) values. Cytotoxicity experiments were performed four times.

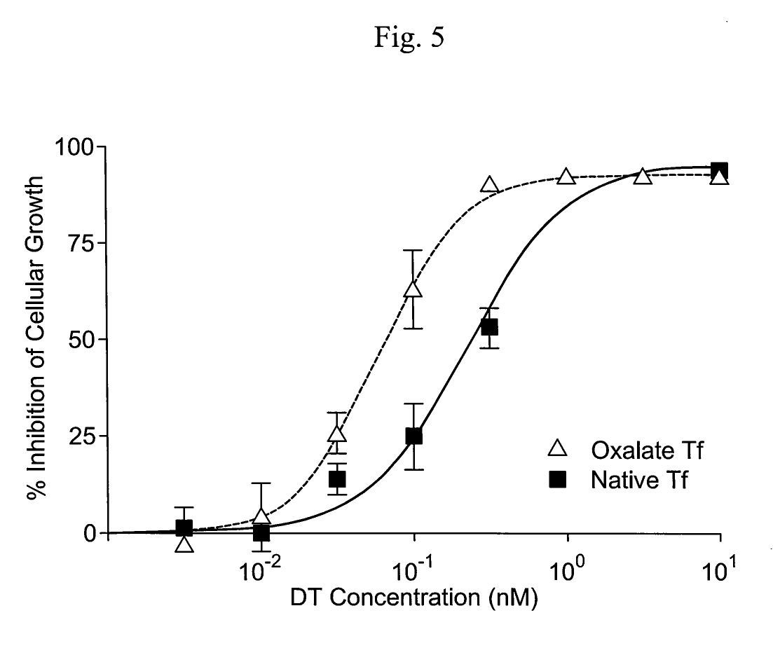

To test whether an increase in cellular association would translate to increased Tf drug carrier efficacy, we produced DT conjugates with oxalate Tf and native Tf. These conjugates were administered to cultured HeLa cells over a range of DT concentrations (10⁻³ nM to 10 nM), and using the MTT assay, the % inhibition of cellular growth was assessed after 48 hours. DT conjugates of oxalate Tf are significantly more cytotoxic than DT conjugates of native Tf (FIG. 5). IC₅₀ values, the concentrations at which 50% inhibition of cellular growth was achieved, are 0.22 nM for native Tf compared to 0.06 nM for oxalate Tf.

HeLa cells were selected for the cytotoxicity assay due to their high expression of transferrin receptors (5.4×10⁵ receptors/cell) (Yazdi and Murphy, Cancer Res. 1994; 54(24):6387-94). In future work, additional cell lines expressing TfR, such as the K562 and HL60 human leukemia cell lines, may be tested to broaden the applicability of the conjugates (Berczi et al., Arch Biochem Biophys. 1993; 300(1):356-63). DT was selected as a cytotoxin since the effective concentration range of DT (IC₅₀˜0.1 nM) (Johnson et al., J Biol Chem. 1988; 263(3): 1295-300) is consistent with the concentration range in which increases in cellular association were observed in the cellular trafficking assay (0.1 and 1 nM, FIG. 4). This is in contrast to cytotoxins such as adriamycin, whose effective concentration range is considerably higher (IC₅₀˜1 μM) (Berczi et al., Arch Biochem Biophys. 1993; 300(1):356-63). Thus, oxalate Tf conjugates with these cytotoxins would not be expected to display increased efficacy, since differences in cellular association between oxalate Tf and native Tf diminish as concentrations rise to 10 nM (FIG. 4C). In the results of our cytotoxicity assay, oxalate Tf conjugates of DT showed greater cytotoxicity than native Tf conjugates of DT against HeLa cells, with an IC₅₀ value of 0.06 nM compared to 0.22 nM for native Tf conjugates.

### Example 4

This example demonstrates that mutant transferrin protein with reduced iron release kinetics demonstrate increased cellular association.

The role of iron release inhibition as a method of improving the drug carrier efficacy of transferrin (Tf) was examined for recombinant Tf mutants with varying degrees of iron release inhibition. Three different mutational variants of a recombinant form of Tf, otherwise known as N-His hTf NG were examined for increased cellular association. Compared to native Tf, N-His hTf NG is tagged at its N-terminus with a string of six histidine residues, and the normal glycosylation pattern found in the native protein is absent.

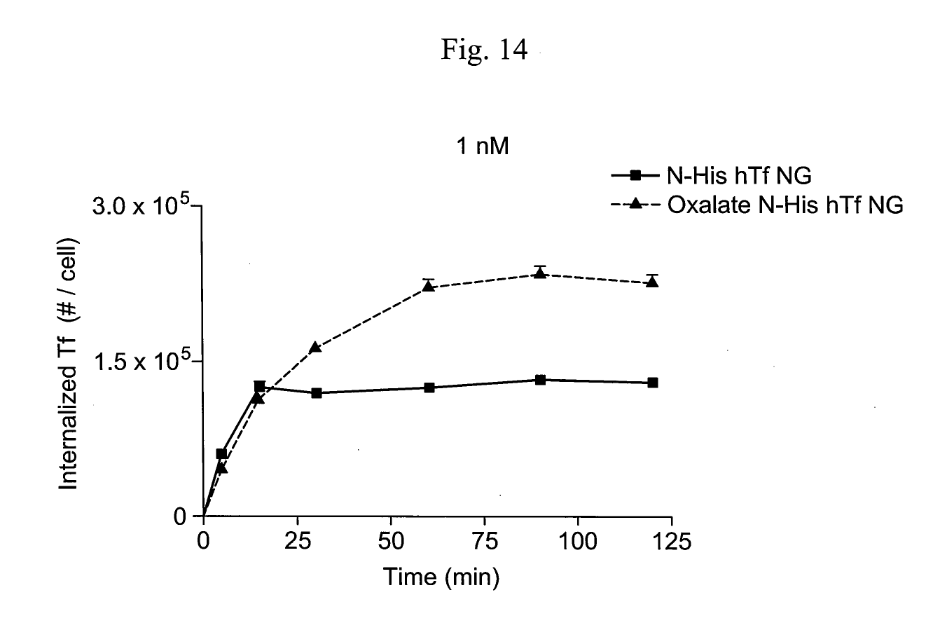

To show that oxalate substitution had the same effect on cellular association for the recombinant Tf as seen previously, cellular trafficking experiments were performed using N-His hTf NG and its oxalate counterpart at a 1 nM concentration with HeLa cells (FIG. 14). Comparing the area under the curve (AUC) values for the internalized Tf/cell vs. time plots, the oxalate N-His hTf NG (2.3×10⁷ molecules/cell) displayed a 58% increase over N-His hTf NG (1.4×10⁷ molecules/cell). This compared favorably to the 57% increase in AUC observed in Example 2 at a 1 nM·Tf concentration.

After determining that the recombinant Tf performs similarly to native Tf, cellular trafficking experiments were performed with three recombinant Tf mutants with decreased iron release kinetics. The first mutant, N-His K206E hTf NG, contains a lysine (K) to glutamic acid (E) mutation at residue 206 of the recombinant Tf molecule. This mutation occurs at a key residue near the iron binding site of the N-terminal lobe of Tf involved in the facilitation of iron release at endosomal pH. This lysine to glutamic acid mutation is able to reduce the iron release rate of the N-terminal lobe iron binding site. The other two Tf mutants were N-His K206E/R632A hTf NG and N-His K206E/K534A hTf NG. Both Tf mutants contain amino acid mutations in proximity to the iron binding sites of both the N-terminal and C-terminal lobes. N-His K206E/R632A hTf NG contains an arginine (R) to alanine (A) mutation at residue 632 of the recombinant Tf molecule in addition to the N-terminal lobe mutation found in N-His K206E hTf NG. This arginine to alanine mutation occurs at a key residue near the iron binding site of the C-terminal lobe of Tf, which acts to reduce the iron release rate of the C-terminal lobe iron binding site. N-His K206E/K534A hTf NG contains a lysine (K) to alanine (A) mutation at residue 534 of the recombinant Tf molecule in addition to the N-terminal lobe mutation found in N-His K206E hTf NG. This lysine to alanine mutation also occurs at a key residue near the iron binding site of the C-terminal lobe, which is also able to reduce the iron release rate of the C-terminal lobe iron binding site. Therefore, while the N-His K206E hTf NG mutant exhibits reduced iron release in only one of its two iron binding sites, the other two mutants exhibit reduced iron release at both sites.

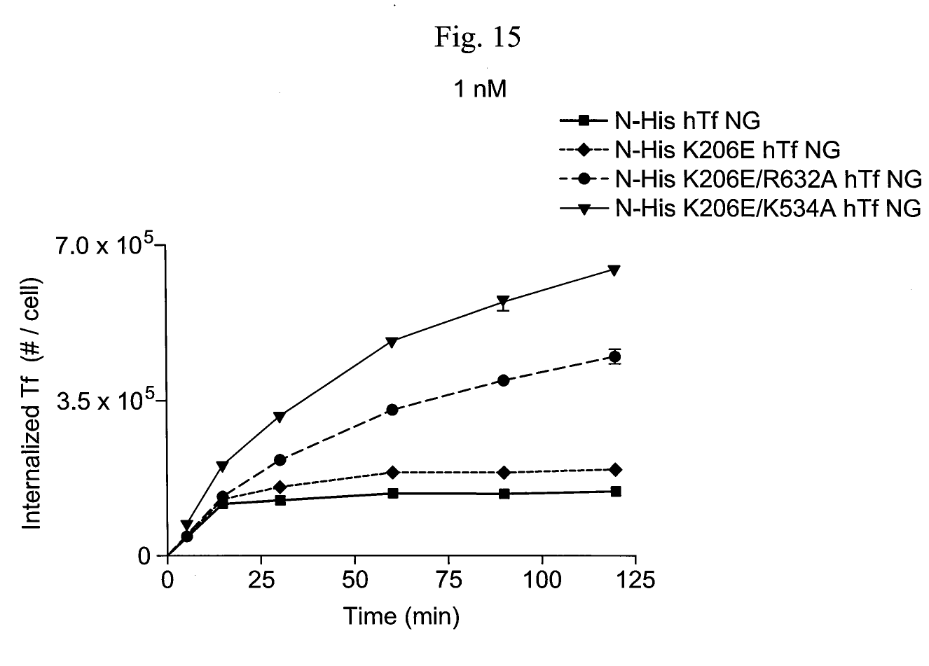

As expected, cellular trafficking experiments performed at a 1 nM Tf concentration with HeLa cells demonstrated that the two mutants with reduced iron release at both iron binding sites have an increased cellular association (FIG. 15). Comparing the area under the curve (AUC) values of their cellular association curves, N-HisK206E hTf NG (2.0×10⁷ molecules/cell), N-His K206E/R632A hTf NG (3.6×10⁷ molecules/cell), and N-His K206E/K534A hTf NG (5.1×10⁷ molecules/cell) displayed a 27%, 129%, and 231% increase over N-His hTf NG (1.6×10⁷ molecules/cell), respectively. Accordingly, the increased cellular association of the recombinant Tf mutants should also result in an improved drug carrier efficacy in cell cytotoxicity studies, as seen for oxalate bound Tf complexes. Thus, Tf mutants that display reduced iron release kinetics can be used for improved cancer drug delivery.

### REFERENCES

1. Cazzola M, Bergamaschi G, Dezza L, Arosio P. Manipulations of cellular iron metabolism for modulating normal and malignant cell proliferation: achievements and prospects. Blood. 1990; 75(10):1903-19.
2. Reizenstein P. Iron, free radicals and cancer. Med Oncol Tumor Pharmacother. 1991; 8(4):229-33.
3. Saul J M, Annapragada A V, Bellamkonda R V. A dual-ligand approach for enhancing targeting selectivity of therapeutic nanocarriers. J Control Release. 2006; 114(3):277-87.
4. Kreitman R J. Immunotoxins for targeted cancer therapy. Aaps J. 2006; 8(3):E532-51.
5. Lim C J, Shen W C. Transferrin-oligomers as potential carriers in anticancer drug delivery. Pharm Res. 2004; 21(11):1985-92.
6. Lai H, Sasaki T, Singh NP, Messay A. Effects of artemisinin-tagged holotransferrin on cancer cells. Life Sci. 2005; 76(11):1267-79.
7. Johnson V G, Wilson D, Greenfield L, Youle R J. The role of the diphtheria toxin receptor in cytosol translocation. J Biol Chem. 1988; 263(3):1295-300.
8. Beyer U, Rothem-Rutishauser B, Unger C, Wunderli-Allenspach H, Kratz F. Differences in the intracellular distribution of acid-sensitive doxorubicin-protein conjugates in comparison to free and liposomal formulated doxorubicin as shown by confocal microscopy. Pharm Res. 2001; 18(1):29-38.
9. Hu-Lieskovan S, Heidel J D, Bartlett D W, Davis M E, Triche T J. Sequence-specific knockdown of EWS-FLI1 by targeted, nonviral delivery of small interfering RNA inhibits tumor growth in a murine model of metastatic Ewing's sarcoma. Cancer Res. 2005; 65(19):8984-92.
10. Tros De Ilarduya C, Bunuales M, Qian C, Duzgunes N. Antitumoral activity of transferrin-lipoplexes carrying the IL-12 gene in the treatment of colon cancer. J Drug Target. 2006; 14(8):527-35.
11. Maruyama K, Ishida O, Kasaoka S, Takizawa T, Utoguchi N, Shinohara A, Chiba M, Kobayashi H, Eriguchi M, Yanagie H. Intracellular targeting of sodium mercaptoundecahydrododecaborate (BSH) to solid tumors by transferrin-PEG liposomes, for boron neutron-capture therapy (BNCT). J Control Release. 2004; 98(2): 195-207.
12. Chiu S J, Liu S, Perrotti D, Marcucci G, Lee R J. Efficient delivery of a Bcl-2-specific antisense oligodeoxyribonucleotide (G3139) via transferrin receptor-targeted liposomes. J Control Release. 2006; 112(2): 199-207.
13. Weaver M, Laske D W. Transferrin receptor ligand-targeted toxin conjugate (Tf-CRM107) for therapy of malignant gliomas. J Neurooncol. 2003; 65(1):3-13.
14. Yazdi P T, Wenning L A, Murphy R M. Influence of cellular trafficking on protein synthesis inhibition of immunotoxins directed against the transferrin receptor. Cancer Res. 1995; 55(17):3763-71.
15. Yazdi P T, Murphy R M. Quantitative analysis of protein synthesis inhibition by transferrin-toxin conjugates. Cancer Res. 1994; 54(24):6387-94.
16. Wenning L A, Yazdi P T, Murphy R M. Quantitative analysis of protein synthesis inhibition and recovery in CRM107 immunotoxin-treated HeLa cells. Biotechnol Bioeng. 1998; 57(4):484-96.
17. Sarkar C A, Lowenhaupt K, Horan T, Boone T C, Tidor B, Lauffenburger D A. Rational cytokine design for increased lifetime and enhanced potency using pH-activated “histidine switching” Nat Biotechnol. 2002; 20(9):908-13.
18. Hinton P R, Johlfs M G, Xiong J M, Hanestad K, Ong K C, Bullock C, Keller S, Tang M T, Tso J Y, Vasquez M, Tsurushita N. Engineered human IgG antibodies with longer serum half-lives in primates. J Biol Chem. 2004; 279(8):6213-6.
19. Ciechanover A, Schwartz A L, Dautry-Varsat A, Lodish H F. Kinetics of internalization and recycling of transferrin and the transferrin receptor in a human hepatoma cell line. Effect of lysosomotropic agents. J Biol Chem. 1983; 258(16):9681-9.
20. French A R, Lauffenburger D A. Controlling receptor/ligand trafficking: effects of cellular and molecular properties on endosomal sorting. Ann Biomed Eng. 1997; 25(4):690-707.
21. Hentze M W, Muckenthaler M U, Andrews N C. Balancing acts: molecular control of mammalian iron metabolism. Cell. 2004; 117(3):285-97.
22. Bali P K, Zak O, Aisen P. A new role for the transferrin receptor in the release of iron from transferrin. Biochemistry. 1991; 30(2):324-8.
23. Lebron J A, Bennett M J, Vaughn D E, Chirino A J, Snow P M, Mintier G A, Feder J N, Bjorkman P J. Crystal structure of the hemochromatosis protein HFE and characterization of its interaction with transferrin receptor. Cell. 1998; 93(1): 111-23.
24. Rao B M, Lauffenburger D A, Wittrup K D. Integrating cell-level kinetic modeling into the design of engineered protein therapeutics. Nat Biotechnol. 2005; 23(2):191-4.
25. Halbrooks P J, Mason A B, Adams T E, Briggs S K, Everse S J. The oxalate effect on release of iron from human serum transferrin explained. J Mol Biol. 2004; 339(1):217-26.
26. Costello J, Landwehr D M. Determination of oxalate concentration in blood. Clin Chem. 1988; 34(8):1540-4.
27. Berczi A, Barabas K, Sizensky J A, Faulk W P. Adriamycin conjugates of human transferrin bind transferrin receptors and kill K562 and HL60 cells. Arch Biochem Biophys. 1993; 300(1):356-63.
28. Gerweck L E, Vijayappa S, Kozin S. Tumor pH controls the in vivo efficacy of weak acid and base chemotherapeutics. Mol Cancer Ther. 2006; 5(5): 1275-9.
29. Giannetti A M, Halbrooks P J, Mason A B, Vogt T M, Enns C A, Bjorkman P J. The molecular mechanism for receptor-stimulated iron release from the plasma iron transport protein transferrin. Structure (Camb). 2005; 13(11):1613-23.
30. Johnson M B, Enns C A. Diferric transferrin regulates transferrin receptor 2 protein stability. Blood. 2004; 104(13):4287-93.
31. Aloj L, Jogoda E, Lang L, Caraco C, Neumann R D, Sung C, Eckelman W C. Targeting of transferrin receptors in nude mice bearing A431 and LS174T xenografts with [18F]holo-transferrin: permeability and receptor dependence. J Nucl Med. 1999; 40(9):1547-55.
32. Anthony D A L, French R. Intracellular receptor/ligand sorting based on endosomal retention components. Biotechnology and Bioengineering. 1996; 51(3):281-297.
33. Cheng Y, Zak O, Aisen P, Harrison S C, Walz T. Structure of the human transferrin receptor-transferrin complex. Cell. 2004; 116(4):565-76.
34. Marsh M, Griffiths G, Dean G E, Mellman I, Helenius A. Three-dimensional structure of endosomes in BHK-21 cells. Proc Natl Acad Sci USA. 1986; 83(9):2899-903.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An anti-cancer therapeutic comprising an anti-cancer agent conjugated to a transferrin (Tf) molecule, wherein said Tf molecule has reduced iron release kinetics as compared to wild type Tf.

2. The anti-cancer therapeutic of claim 1, wherein said Tf is bound to an anion other than carbonate.

3. The anti-cancer therapeutic of claim 2, wherein said anion is oxalate.

4. The anti-cancer therapeutic of claim 1, wherein said Tf comprises a mutation that results in reduced iron release kinetics.

5. The anti-cancer therapeutic of claim 4, wherein the amino acid sequence of said Tf molecule is at least 85% identical to the amino acid sequence of SEQ ID NO:1.

6. The anti-cancer therapeutic of claim 5, wherein said Tf molecule further comprises at least one mutation of a residue selected from the group consisting of K206, K296, H349, H350, K534, R632, D634, and combinations thereof.

7. The anti-cancer therapeutic of claim 1, wherein said anti-cancer agent is a diphtheria toxin.

8. The anti-cancer therapeutic of claim 7, wherein said diphtheria toxin contains a mutation that reduces non-specific cell-binding.

9. The anti-cancer therapeutic of claim 8, wherein said diphtheria toxin is CRM107.

10. A method of treating cancer in a mammal, comprising administering an anti-cancer therapeutic of claim 1 to a mammal with cancer.

11. The method of claim 10, wherein said mammal is a human.

12. The method of claim 10, wherein said cancer is brain cancer.

13. The method of claim 12, wherein said cancer comprises a glioblastoma multiforme tumor.

14. The method of claim 10, wherein said method further comprises administering an adjuvant cancer therapy.

15. The method of claim 14, wherein said adjuvant cancer therapy is radiotherapy.

16. A pharmaceutical composition comprising an anti-cancer therapeutic of claim 1.

17. The pharmaceutical composition of claim 16, wherein said composition further comprises a physiologically acceptable carrier and a pharmaceutically acceptable auxiliary substance.


SEQUENCE LISTING (1 sequence)

SEQ ID NO: 1 - protein, 698 aa, Homo sapiens
  human serum iron transport protein transferrin, granulocyte/pollen-binding protein (GPBP), PRO1557 protein, PRO2086 protein
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP